(19) United States
(12) Patent Application Publication
(10) Pub. No.: US 2023/0404911 A1
(43) Pub. Date: Dec. 21, 2023

(54) DRUG DELIVERY SYSTEM FOR ULTRA-LOW DOSE ESTROGEN COMBINATIONS AND METHODS AND USES THEREOF

(71) Applicant: LUPIN INC., Baltimore, MD (US)

(72) Inventors: Vinita GUPTA, Naples, FL (US); Richard HOLL, Somerset, NJ (US); James GAREGNANI, Somerset, NJ (US); Gregory KAUFMAN, Somerset, NJ (US)

(21) Appl. No.: 18/240,284

(22) Filed: Aug. 30, 2023

Related U.S. Application Data
(63) Continuation of application No. 17/878,709, filed on Aug. 1, 2022, now abandoned.
Application No. 17/878,709 is a continuation-in-part of application No. 16/695,888, filed on Nov. 26, 2019, now abandoned.
(60) Provisional application No. 62/778,090, filed on Dec. 11, 2018.

(51) Int. Cl. A61K 9/00; A61P 15/18; A61K 31/566; A61K 47/32

(52) U.S. Cl. CPC A61K 9/0036; A61P 15/18; A61K 31/566; A61K 47/32

(57) ABSTRACT

The invention relates to a drug delivery system for contraception including a steroidal estrogenic compound, a steroidal progestogenic compound or a combination thereof for the purposes of contraception in a subject, and provides for an ultra-low dose delivery of a steroidal estrogenic compound which minimizes or eliminates a burst release effect. The invention also relates to a method of contraception using the drug delivery system and method of manufacturing the drug delivery system.

### CROSS-REFERENCE TO RELATED APPLICATIONS

This patent application is a Continuation of U.S. patent application Ser. No. 17/878,709, filed Aug. 1, 2022, which was a Continuation-in-Part of U.S. application Ser. No. 16/695,888, filed Nov. 26, 2019, which claims priority to U.S. Provisional Application No. 62/778,090, filed Dec. 11, 2018, the contents of each of which is hereby incorporated by reference herein in its entirety.

### FIELD OF THE INVENTION

The invention relates to reproductive medicine, for instance, to female contraception. The invention also relates to a drug delivery system for release of a steroidal estrogenic compound, a steroidal progestogenic compound or a combination thereof; a method of manufacturing the drug delivery system; and a method of contraceptive treatment in animals, such as mammals (including, without limitation, humans), using the drug delivery system.

### BACKGROUND

The following comprises information that may be useful in understanding the present invention. It is not an admission that any of the information, publications or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

A variety of chemical and mechanical methods for controlling fertility and for preventing pregnancy are known. Approaches such as sterilization, the use of condoms, intrauterine devices, spermicidal creams and jellies, foam tablets, and oral pills are currently available as a prevention against pregnancy. These methods, though effective to a variable extent, also have limitations. Most of the devices require constant motivation on the part of the user and some approaches such as sterilization and intrauterine devices require specialized medical attention. The oral pill is a popular method of contraception but the oral contraceptives have many undesirable side effects and require the daily ingestion of a tablet. The use of the vaginal ring as a means of administering effective contraceptive steroids through a vaginal route is a means of overcoming some of these drawbacks. For vaginal rings, the concentration of contraceptive agents included in the vaginal rings may pose problems for patients due to adverse side effects associated with high concentration of contraceptive agents.

Estrogen and progestin are molecular classes of two female sex hormones that are used for contraceptive purposes. Combinations of estrogen and progestin work by establishing a contraceptive effect, such as, for example, preventing ovulation (the release of eggs from the ovaries) in a subject; changing the subject's lining of the uterus (womb) to prevent pregnancy from developing; changing the subject's mucus at the cervix (opening of the uterus) to prevent sperm (male reproductive cells) from entering; and/or any other medical condition(s) in the subject that prevents pregnancy.

Combined estrogen-progestin oral contraceptives provide reliable contraception as well as several non-contraceptive benefits. Estrogen- and progestin-based contraceptives, however, have several well-known side effects. Adverse side effects include breast tenderness, nausea, bloating, unscheduled bleeding, weight gain, increased cardiovascular risks, such as, myocardial infarction, hypertension, stroke, and increased risk of venous thromboembolic events.

There have been many attempts to develop low-dose and ultra-low dose estrogen combination oral contraceptives using ethinyl estradiol, a potent estrogen, to reduce the side effects of the estrogen but oral contraceptive have limitations due to adverse side effects and the need to regularly (typically daily) ingest the contraceptive. Current low-dose combination oral contraceptives are defined as orally-administered drug products that contain 35 micrograms or less of ethinyl estradiol intended for once per day administration. Examples of approved and marketed ethinyl estradiol combination products include Yasmin® (drospirenone/ethinyl estradiol tablets) marketed by Bayer, Levora® (levonorgestrel/ethinyl estradiol tablets) marketed by Mayne Pharma, and Ortho-Tri-Cyclen® Lo (norgestimate/ethinyl estradiol tablets) marketed by Janssen.

Reference ultra-low dose combination oral contraceptives are generally contain 20 micrograms or less of ethinyl estradiol intended for once per day administration. Examples of approved and marketed combination oral contraceptive include Yaz® (drospirenone/ethinyl estradiol tablets) marketed by Bayer and Lo Loestrin® Fe (norethindrone acetate/ethinyl estradiol) marketed by Allergan.

An alternative to oral contraceptives is a vaginal drug delivery system. Such systems, for example, are described in U.S. Pat. Nos. 3,995,633 and 3,995,634, where separate, preferably spherical or cylindrical, reservoirs containing different active substances are assembled in specially constructed holders. A vaginal drug delivery system is also described in U.S. Pat. No. 4,237,885, where a tube or coil of polymeric material is divided into portions by means of a plurality of “spacers” provided within the tube. Each of the separate tube portions is filled with a different active substance in a silicone fluid and the two ends of the tube are subsequently connected to one another. In this vaginal drug delivery system, however, transport (diffusion) of active material from one reservoir to the other takes place through the wall of the tube, especially upon prolonged storage, such that the pre-set fixed release ratio between the active substances in question changes or fluctuates for a period of time. This diffusion can have unintended consequences on a patient's health, and creates an unpredictable system with decreased efficacy and a short storage period.

A two-layered vaginal ring system is described in European Patent Publication No. 0050867. In this system, the ring comprises a pharmacologically acceptable supporting ring covered by two layers preferably of silicone elastomers where the inner layer is a silicone elastomer loaded with an active substance. A similar ring shaped vaginal delivery system is described in U.S. Pat. No. 4,292,965.

In U.S. Pat. No. 4,596,576, a two-compartment vaginal ring is disclosed, where each compartment contains a different active substance. To achieve a suitable ring with a constant release ratio between the various active substances, it was necessary, however, to join the end portions of the compartments by inert stoppers, preferably glass stoppers which can pose a problem during administration of the ring to a subject and in instances where the ring is retained within the subject's vaginal tract.

PCT International Patent Publication No. WO 97/02015 discloses a two-compartment device including a first compartment consisting of a core, a medicated middle layer, and a non-medicated outer layer and a second compartment consisting of a medicated core and a non-medicated outer layer. This structure, however, is complicated and difficult to manufacture.

Another contraceptive intravaginal ring that is currently approved and marketed in the United States is NuvaRing®. NuvaRing® contains 11.7 mg of etonogestrel and 2.7 mg of ethinyl estradiol. After administration, NuvaRing® appears to release about 120 micrograms per day of etonogestrel and about 15 micrograms per day of ethinyl estradiol for a period of 3 weeks (or 21 days). U.S. Pat. No. 5,989,581 discloses a fixed dose combination of progestin compound (like norethindrone) and estrogen compound (like ethinyl estradiol).

A contraceptive vaginal ring releasing norethindrone acetate and ethinyl estradiol was disclosed in a 1994 journal article that comprises a core design contraceptive vaginal ring releasing 650 micrograms of norethindrone acetate and 10, 20, 30 or 65 micrograms of ethinyl estradiol daily (Ballagh et al., “A Contraceptive Vaginal Ring Releasing Norethindrone Acetate and Ethinyl Estradiol,” Contraception, 50(6):517-533 (1994)). This ring was developed and tested in 99 women. Two combined contraceptive vaginal rings each releasing approximately 1 mg norethindrone acetate and either 20 micrograms or 15 micrograms of ethinyl estradiol for 24 hours were tested at three clinic sites (Wiseberg et al., “A Comparative Study of Two Contraceptive Vaginal Rings Releasing Norethindrone Acetate and Differing Doses of Ethinyl Estradiol,” Contraception, 59(5):305-310 (1999)).

United States Food and Drug Administration (FDA) has recently approved a contraceptive vaginal ring product, namely, Annovera®. Annovera® is a silicone elastomer vaginal system containing 103 milligrams (mg) of segesterone acetate and 17.4 mg of ethinyl estradiol. Annovera® releases on a mean average 0.15 mg/day of segesterone acetate and 0.013 mg/day of ethinyl estradiol.

One oral combination contraceptive containing estradiol (as the hemihydrate) is approved and marketed in the European Union as Zoely®. Estradiol, however, has low oral bioavailability (2% to 10%) due to first-pass metabolism. Zoely® contains a fixed dose combination of 2.5 mg of nomegestrol acetate and 1.5 mg of estradiol (as the hemihydrate). For Zoely® contraceptive efficacy was achieved with average serum estradiol concentrations of 50 picograms/ml (pg/ml).

Another presently commercialized product at the time of filing this disclosure containing estradiol is Estring®. Estring® is an intravaginal ring that contains 2 mg of estradiol and indicated for the treatment of moderate to severe symptoms of vulvar and vaginal atrophy due to menopause. After administration, Estring® releases around 7.5 microgram of estradiol per day for a period of 90 days. The mean steady state serum estradiol concentration upon administration of Estring® estimates of 7.8, 7.0, 7.0, and 8.1 picograms/mL at weeks 12, 24, 36, and 48, respectively.

### SUMMARY OF THE INVENTION

The inventions described and claimed herein have many attributes and embodiments including, but not limited to, those set forth or described or referenced in this Summary. It is not intended to be all-inclusive and the inventions described and claimed herein are not limited to or by the features or embodiments identified in this introduction, which is included for purposes of illustration only and not restriction.

It is an object of the invention to provide a drug delivery system for contraception including a steroidal estrogenic compound, a steroidal progestogenic compound or a combination thereof for the purposes of contraception in a subject, and provides for an ultra-low dose delivery of a steroidal estrogenic compound. The invention also relates to a method of contraception using the drug delivery system and method of manufacturing the drug delivery system.

In some aspects, this disclosure provides for a drug delivery system comprising a core comprising a steroidal estrogenic compound and a steroidal progestogenic compound. The core of the system is, optionally, covered by a membrane or a skin. The drug delivery system is such that when it is placed in a subject's vaginal tract, it delivers a therapeutically effective dose steroidal estrogenic compound and a therapeutically effective dose of the steroidal progestogenic compound to the subject (optionally, at a controlled rate) for a period of time (or total period of time) to produce a contraceptive effect in the subject. In some aspects, the therapeutically effective dose of steroidal estrogenic compound is an ultra-low dose.

In some aspects, the invention is directed to a method of contraception. This method comprises a step of retainably positioning the drug-delivery system within a subject's vaginal tract; retaining the system within the subject's vaginal tract for a period of time; and removing the system from the subject after the period of time. In some embodiments, a method of contraception for a total period of time, which comprises (a) retainably positioning the drug-delivery system disclosed in this application within a subject's vaginal tract and retaining the system within the subject's vaginal tract for a first period of time; (b) removing the system from the subject's vaginal tract after the first period of time; (c) reinserting the system within the subject's vaginal tract after a second period of time and retaining the system within the subject's vaginal tract for a third period of time; (d) removing the system from the subject's vaginal tract after the third period of time; (e) optionally reinserting the system within the subject's vaginal tract after the third period of time, retaining the system within the subject's vaginal tract for a fourth period of time, and removing the system from the subject's vaginal tract after the fourth period of time; and (f) optionally repeating step (e) until the end of the total period of time. In some embodiments, the first period of time and third period of time are the same amount of time or different amounts of time. In some embodiments, the second period of time and fourth period of time are the same amount of time or different amounts of time.

In some aspects, the invention is directed to a method of manufacturing the drug delivery system disclosed in this application, the method of manufacturing comprising (a) producing the core comprising the combination of the steroidal estrogenic compound and the steroidal progestogenic compound, and (b) covering a portion or all of the core with the membrane to form the drug delivery system. In some aspects, the core is coextruded with the membrane to form the drug delivery system.

In some aspects, the invention is directed to a method of making the drug delivery system disclosed in this application. This method comprises a step of producing a core comprising the combination of a steroidal estrogenic compound and a steroidal progestogenic compound and a subsequent step of covering a portion or all of the core with a membrane to form the drug delivery system.

In some aspects, the invention is directed to a drug delivery system for use in contraception by administering the system to a subject's vaginal tract, said system comprising (a) a core comprising a steroidal estrogenic compound and a steroidal progestogenic compound; (b) a membrane covering a portion or all of the core; wherein, when the system is in the subject's vaginal tract, the system delivers a therapeutically effective dose of the steroidal estrogenic compound and a therapeutically effective dose of the steroidal progestogenic compound to the subject for a period of time to produce a contraceptive effect in the subject, and the system delivers less than 0.013 mg per day of the steroidal estrogenic compound to the subject's vaginal tract.

In some aspects, the period of time is about 21 days, about 24 days, about 26 days, or about 3 months after administration to the subject's vaginal tract.

In some aspects, the drug delivery system delivers the therapeutically effective dose of the steroidal estrogenic compound and the therapeutically effective dose of steroidal progestogenic compound to the subject at a controlled rate for the period of time, and the period of time is about 21 days, about 24 days, about 26 days, or about 3 months after administration to the subject's vaginal tract.

In some aspects, the steroidal estrogenic compound is ethinyl estradiol or estradiol.

In some aspects, the drug delivery system delivers about 0.010 mg per day of ethinyl estradiol to the subject for about 21 days, about 24 days, about 26 days, or about 3 months after administration to the subject's vaginal tract.

In some aspects, the steroidal progestogenic compound is selected from a group consisting of drospirenone, norethindrone, norethindrone acetate, levonorgestrel, etonogestrel, and norgestimate.

In some aspects, the steroidal progestogenic compound is etonogestrel and the steroidal estrogenic compound is ethinyl estradiol.

In some aspects, the amount of etonogestrel in the drug delivery system is about 11.7 mg and the amount of ethinyl estradiol in the drug delivery system is about 1.8 mg, or the amount of etonogestrel in the drug delivery system is about 35.1 mg and the amount of ethinyl estradiol in the drug delivery system is about 5.4 mg.

In some aspects, the drug delivery system releases on an average about 0.12 mg of etonogestrel per day for about 21 days, about 24 days, about 26 days, or about 3 months after administration of the system to the subject's vaginal tract.

In some aspects, the drug delivery system releases on an average about 0.010 mg of ethinyl estradiol per day for about 21 days, about 24 days, about 26 days, or about 3 months after administration of the system to the subject's vaginal tract.

In some aspects, the steroidal progestogenic compound is norethindrone acetate and the steroidal estrogenic compound is ethinyl estradiol.

In some aspects, the amount of norethindrone acetate in the drug delivery system is about 100 mg and the amount of ethinyl estradiol in the drug delivery system is about 1.8 mg, or the amount of norethindrone acetate in the drug delivery system is about 300 mg and the amount of ethinyl estradiol in the drug delivery system is about 5.4 mg.

In some aspects, the drug delivery system releases on an average about 1 mg of norethindrone acetate per day for about 21 days, about 24 days, about 26 days, or about 3 months after administration of the drug delivery system to the subject's vaginal tract.

In some aspects, the drug delivery system releases on an average about 0.010 mg of ethinyl estradiol per day for about 21 days, about 24 days, about 26 days, or about 3 months after administration of the drug delivery system to the subject's vaginal tract.

In some aspects, the steroidal progestogenic compound is etonogestrel and the steroidal estrogenic compound is estradiol.

In some aspects, the amount of etonogestrel in the drug delivery system is about 11.7 mg and the amount of estradiol in the drug delivery system is about 27 mg, or the amount of etonogestrel in the drug delivery system is about 35.1 mg and the amount of estradiol in the drug delivery system is about 81 mg.

In some aspects, the drug delivery system releases on an average about 0.12 mg of etonogestrel per day for about 21 days, about 24 days, about 26 days, or about 3 months after administration of the drug delivery system to the subject's vaginal tract.

In some aspects, the drug delivery system releases on an average about 0.15 mg of estradiol per day for about 21 days, about 24 days, about 26 days, or about 3 months after administration of the drug delivery system to the subject's vaginal tract.

In some aspects, steroidal progestogenic compound is norethindrone acetate and the steroidal estrogenic compound is estradiol.

In some aspects, the amount of norethindrone acetate in the drug delivery system is about 100 mg and the amount of estradiol in the drug delivery system is about 27 mg, or the amount of norethindrone acetate in the drug delivery system is about 300 mg and the amount of estradiol in the drug delivery system is about 81 mg.

In some aspects, the drug delivery system releases on an average about 1 mg of norethindrone acetate per day for about 21 days, about 24 days, about 26 days, or about 3 months after administration of the drug delivery system to the subject's vaginal tract.

In some aspects, the drug delivery system releases on an average about 0.15 mg of estradiol per day for about 21 days, about 24 days, about 26 days, or about 3 months after administration of the drug delivery system to the subject's vaginal tract.

In some aspects, the steroidal progestogenic compound is levonorgestrel or drospirenone and the steroidal estrogenic compound is ethinyl estradiol.

In some aspects, the amount of levonorgestrel in the drug delivery system is about 49 mg and the amount of ethinyl estradiol in the drug delivery system is about 1.8 mg, or the amount of levonorgestrel in the drug delivery system is about 56 mg and the amount of ethinyl estradiol in the drug delivery system is about 2.1 mg, or the amount of levonorgestrel in the drug delivery system is about 60 mg and the amount of ethinyl estradiol in the drug delivery system is about 2.2 mg, or the amount of levonorgestrel in the drug delivery system is about 146 mg and the amount of ethinyl estradiol in the drug delivery system is about 5.4 mg.

In some aspects, the drug delivery system releases on an average about 0.50 mg of levonorgestrel per day for about 21 days, about 24 days, about 26 days, or about 3 months after administration of the system to the subject's vaginal tract.

In some aspects, the drug delivery system releases on an average about 0.010 mg of ethinyl estradiol per day for about 21 days, about 24 days, about 26 days, or about 3 months after administration of the system to the subject's vaginal tract.

In some aspects, the amount of drospirenone in the drug delivery system is about 300 mg and the amount of ethinyl estradiol in the drug delivery system is about 1.8 mg, or the amount of drospirenone in the drug delivery system is about 343 mg and the amount of ethinyl estradiol in the drug delivery system is about 2.1 mg, or the amount of drospirenone in the drug delivery system is about 371 mg and the amount of ethinyl estradiol in the drug delivery system is about 2.2 mg.

In some aspects, the drug delivery system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

In some aspects, the drug delivery system releases on an average about 0.010 mg of ethinyl estradiol per day for about 21 days, about 24 days, or about 26 days after administration of the system to the subject's vaginal tract.

In some aspects, the steroidal progestogenic compound is levonorgestrel or drospirenone and the steroidal estrogenic compound is estradiol.

In some aspects, the amount of levonorgestrel in the drug delivery system is about 49 mg and the amount of estradiol in the drug delivery system is about 27 mg, or the amount of levonorgestrel in the drug delivery system is about 56 mg and the amount of estradiol in the drug delivery system is about 31 mg, or the amount of levonorgestrel in the drug delivery system is about 60 mg and the amount of estradiol in the drug delivery system is about 33 mg, or the amount of levonorgestrel in the drug delivery system is about 146 mg and the amount of estradiol in the drug delivery system is about 81 mg.

In some aspects, the drug delivery system releases on an average about 0.5 mg of levonorgestrel per day for about 21 days, about 24 days, about 26 days, or about 3 months after administration of the system to the subject's vaginal tract.

In some aspects, the drug delivery system releases on an average about 0.15 mg of estradiol per day for about 21 days, about 24 days, about 26 days, or about 3 months after administration of the system to the subject's vaginal tract.

In some aspects, the amount of drospirenone in the drug delivery system is about 300 mg and the amount of estradiol in the drug delivery system is about 27 mg, or the amount of drospirenone in the drug delivery system is about 343 mg and the amount of estradiol in the drug delivery system is about 31 mg, or the amount of drospirenone in the drug delivery system is about 371 mg and the amount of estradiol in the drug delivery system is about 33 mg.

In some aspects, the drug delivery system releases on an average about 3 mg of drospirenone per day for about 21 days or about 24 days after administration of the system to the subject's vaginal tract, or an average about 1 mg of drospirenone per day for about 26 days after administration of the system to the subject's vaginal tract.

In some aspects, the drug delivery system releases on an average about 0.15 mg of estradiol per day for about 21 days, about 24 days, or about 26 days after administration of the system to the subject's vaginal tract.

In some aspects, the core is made of a thermoplastic polymer or an elastomer.

In some aspects, the core is made of a material selected from the group consisting of low-density polyethylene, ethylene-vinyl acetate copolymers, thermoplastic polyurethane, styrene-butadiene-styrene copolymers, and a combination thereof.

In some aspects, the membrane is made of a material selected from the group consisting of olefins, vinyl polymers, rubber polymers, silicon polymers, microporous polymers, diffusion polymers, polyethylene, ethylene-vinyl acetate copolymers, thermoplastic polyurethanes, polyether-ester polymers, cellulose, and a combination thereof.

In some aspects, the drug delivery system is adapted for administration in the subject's vaginal tract.

In some aspects, the drug delivery system is substantially in the form of a ring, a sphere, a cylinder, an implant, an intrauterine system, a helical coil, a toroid, a spring, or a combination thereof.

In some aspects, the drug delivery system is substantially in the shape of a ring. In some aspects, the drug delivery ring has an outer diameter of from about 50 millimeters to about 60 millimeters, or an outer diameter of from about 52 millimeters to about 56 millimeters. In some aspects, the ring has a cross-sectional diameter of from about 2.5 millimeters to about 5 millimeters.

In some aspects, the steroidal estrogenic compound and the steroidal progestogenic compound are enclosed in separate sections of the core, or enclosed in the same section of the core. In some aspects, the steroidal estrogenic compound and the steroidal progestogenic compound are enclosed in same or separate compartments within the core. In some aspects, the core comprises a fixed ratio of the steroidal estrogenic compound to the steroidal progestogenic compound.

In some aspects, the subject is a mammal or human.

In some aspects, the invention is directed to a drug delivery system for use in contraception. This drug delivery system can comprise: a core comprising ethinyl estradiol and etonogestrel; and a membrane covering a portion or all of the core; where when the system is in a subject's vaginal tract, the system can deliver a therapeutically effective dose of the ethinyl estradiol and a therapeutically effective dose of the etonogestrel to the subject for a period of time to produce a contraceptive effect in the subject, and the system can deliver less than an average of 0.013 mg per day of the ethinyl estradiol compound to the subject's vaginal tract. In some aspects, the period of time is about 21 days, about 24 days, about 26 days, or about 3 months after administration to the subject's vaginal tract. In some aspects, the average is measured after the burst release of the ethinyl estradiol and etonogestrel after administration of the drug delivery system to the subject.

In some aspects, the amount of etonogestrel in the drug delivery system can be from about 1 mg etonogestrel to about 75 mg etonogestrel. In some aspects, the amount of etonogestrel in the drug delivery system can be from about 8 to 9 mg. In some aspects, the amount of etonogestrel in the drug delivery system can be about 8.5 mg. In some aspects, the amount of ethinyl estradiol in the drug delivery system can be from about 0.1 mg to about 7.5 mg. In some aspects, the amount of ethinyl estradiol in the drug delivery system can be about 1.27 mg. In some aspects, the amount of etonogestrel can be about 8.5 mg and the amount of ethinyl estradiol is about 1.27 mg.

In some aspects, the amount of etonogestrel in the drug delivery system can be from 0.4 to 0.7 wt. %. In some aspects, the amount of etonogestrel can be about 0.5 wt. %. In some aspects, the amount of ethinyl estradiol in the drug delivery system can be from 0.05 to 1.0 wt. %. In some aspects, the amount of ethinyl estradiol in the drug delivery system can be about 0.07 wt. %.

In some aspects, the drug delivery system can be configured to release on an average about 0.1 mg to about 0.2 mg of etonogestrel per day for about 21 days, about 24 days, about 26 days, or about 3 months, after the first day of insertion of the drug delivery device to a subject's vaginal tract. In some aspects, the drug delivery system can be configured to release about 0.12 mg of etonogestrel per day for about 21 days, about 24 days, about 26 days, or about 3 months, after the first day of insertion of the drug delivery device to a subject's vaginal tract. In some aspects, the drug delivery system can be configured to release on an average from about 0.001 to about 0.013 mg of ethinyl estradiol per day, after the first day of insertion of the drug delivery device to a subject's vaginal tract. In some aspects, the system can be configured to release on an average about 0.010 mg or less of ethinyl estradiol per day, after the first day of insertion of the drug delivery device to a subject's vaginal tract. In some aspects, the drug delivery system can be configured to release on an average from about 0.001 to about 0.010 mg of ethinyl estradiol per day, after the first day of insertion of the drug delivery device to a subject's vaginal tract t. In some aspects, the drug delivery system can be configured to release on an average about 0.001, 0.002, 0.003, 0.004, 0.005, 0.006, 0.007, 0.008, 0.009, 0.010, 0.011, 0.012, and 0.013 mg of ethinyl estradiol per day, or any value between the aforementioned values, after the first day of insertion of the drug delivery device to a subject's vaginal tract. In some aspects, the drug delivery system can be configured to release on an average about 0.12 mg of etonogestrel per day, and an average of about 0.010 mg of ethinyl estradiol per day, after the first day of insertion of the drug delivery device to a subject's vaginal tract. As used herein, the term “average” is intended to refer to the mean average.

In some aspects, this disclosure provides for a method of contraception for a total period of time, comprising the steps of: (a) retainably positioning the drug-delivery system within a subject's vaginal tract and retaining the system within the subject's vaginal tract for a selected period of time or total period of time; and (b) removing the system from the subject's vaginal tract after the selected period of time or total period of time.

In some aspects, the selected period of time or total period of time can be about 21 days, about 26 days, about 28 days, or about 3 months.

In some aspects, the total period of time can comprise intermittent removal and reinsertion of the drug delivery system at selected intervals for prescribed periods of time. In some aspects, the selected interval of removal can be 2 days within a 28-day menstrual cycle.

In some aspects, the drug delivery system can be configured to release on an average from about 0.001 to about 0.013 mg of ethinyl estradiol per day for about 21 days after administration to the subject's vaginal tract. In some aspects, the drug delivery system can be configured to release on an average from about 0.001 to about 0.013 mg of ethinyl estradiol per day for about 24 days after administration to the subject's vaginal tract. In some aspects, the drug delivery system can be configured to release on an average about from about 0.001 to about 0.013 mg of ethinyl estradiol per day for about 26 days after administration to the subject's vaginal tract. In some aspects, the drug delivery system can be configured to release on an average about from about 0.001 to about 0.013 mg of ethinyl estradiol per day for about 3 months after administration to the subject's vaginal tract. In some aspects, the drug delivery system can be configured to release on an average about 0.001, 0.002, 0.003, 0.004, 0.005, 0.006, 0.007, 0.008, 0.009, 0.010, 0.011, 0.012, and 0.013 mg of ethinyl estradiol per day, or any value between the aforementioned values, for about 21 days after administration to the subject's vaginal tract. In some aspects, the drug delivery system can be configured to release on an average about 0.001, 0.002, 0.003, 0.004, 0.005, 0.06, 0.007, 0.008, 0.009, 0.010, 0.011, 0.012, and 0.013 mg of ethinyl estradiol per day, or any value between the aforementioned values, for about 24 days after administration to the subject's vaginal tract. In some aspects, the drug delivery system can be configured to release on an average about 0.001, 0.002, 0.003, 0.004, 0.005, 0.006, 0.007, 0.008, 0.009, 0.010, 0.011, 0.012, and 0.013 mg of ethinyl estradiol per day, or any value between the aforementioned values, for about 26 days after administration to the subject's vaginal tract. In some aspects, the drug delivery system can be configured to release on an average about 0.001, 0.002, 0.003, 0.004, 0.005, 0.006, 0.007, 0.008, 0.009, 0.010, 0.011, 0.012, and 0.013 mg of ethinyl estradiol per day, or any value between the aforementioned values, for about 3 months after administration to the subject's vaginal tract. In some aspects, the drug delivery system can be configured to release on an average about 0.013 mg per day of ethinyl estradiol to the subject for about 21 days after administration. In some aspects, the drug delivery system can be configured to release on an average about 0.013 mg per day of ethinyl estradiol to the subject for about 24 days after administration. In some aspects, the drug delivery system can be configured to release on an average about 0.013 mg per day of ethinyl estradiol to the subject for about 26 days after administration. In some aspects, the drug delivery system can be configured to release on an average about 0.013 mg per day of ethinyl estradiol to the subject for about 3 months after administration. In some aspects, the drug delivery system can be configured to release on an average about 0.010 mg per day of ethinyl estradiol to the subject for about 21 days after administration. In some aspects, the drug delivery system can be configured to release on an average about 0.010 mg per day of ethinyl estradiol to the subject for about 24 days after administration. In some aspects, the drug delivery system can be configured to release on an average about 0.010 mg per day of ethinyl estradiol to the subject for about 26 days after administration. In some aspects, the drug delivery system can be configured to release on an average about 0.010 mg per day of ethinyl estradiol to the subject for about 3 months after administration. In some aspects, the drug delivery system can be configured to release on an average about 0.1 mg to about 0.2 mg of etonogestrel to the subject for about 21 days after administration. In some aspects, the drug delivery system can be configured to release on an average 0.1 mg to about 0.2 mg of etonogestrel to the subject for about 24 days after administration. In some aspects, the drug delivery system can be configured to release on an average about 0.1 mg to about 0.2 mg of etonogestrel to the subject for about 26 days after administration. In some aspects, the drug delivery system can be configured to release on an average about 0.1 mg to about 0.2 mg of etonogestrel to the subject for about 3 months after administration. In some aspects, the drug delivery system can be configured to release on an average from about 0.1 mg to about 0.15 mg of etonogestrel per day for about 21 days after administration to the subject's vaginal tract. In some aspects, the drug delivery system can be configured to release on an average from about 0.1 mg to about 0.15 mg of etonogestrel per day for about 24 days after administration to the subject's vaginal tract. In some aspects, the drug delivery system can be configured to release on an average from about 0.1 mg to about 0.15 mg of etonogestrel per day for about 26 days after administration to the subject's vaginal tract. In some aspects, the drug delivery system can be configured to release on an average from about 0.1 mg to about 0.15 mg of etonogestrel per day for about 3 months after administration to the subject's vaginal tract. In some aspects, the drug delivery system can be configured to release on an average about 0.10, 0.11, 0.12, 0.13, 0.14, 0.15, 0.16, 0.17, 0.18, 0.19 or 0.20 mg of etonogestrel per day, or any value between the aforementioned values, for about 21 days after administration to the subject's vaginal tract. In some aspects, the drug delivery system can be configured to release on an average about 0.10, 0.11, 0.12, 0.13, 0.14, 0.15, 0.16, 0.17, 0.18, 0.19 or 0.20 mg of etonogestrel per day, or any value between the aforementioned values, for about 24 days after administration to the subject's vaginal tract. In some aspects, the drug delivery system can be configured to release on an average about 0.10, 0.11, 0.12, 0.13, 0.14, 0.15, 0.16, 0.17, 0.18, 0.19 or 0.20 mg of etonogestrel per day, or any value between the aforementioned values, for about 26 days after administration to the subject's vaginal tract. In some aspects, the drug delivery system can be configured to release on an average about 0.10, 0.11, 0.12, 0.13, 0.14, 0.15, 0.16, 0.17, 0.18, 0.19 or 0.20 mg of etonogestrel per day, or any value between the aforementioned values, for about 3 months after administration to the subject's vaginal tract. In some aspects, the drug delivery system can be configured to release on an average about 0.12 mg of etonogestrel per day for about 21 days after administration to the subject's vaginal tract. In some aspects, the drug delivery system can be configured to release on an average about 0.12 mg of etonogestrel per day for about 24 days after administration to the subject's vaginal tract. In some aspects, the drug delivery system can be configured to release on an average about 0.12 mg of etonogestrel per day for about 26 days after administration to the subject's vaginal tract. In some aspects, the drug delivery system can be configured to release on an average about 0.12 mg of etonogestrel per day for about 3 months after administration to the subject's vaginal tract. In some aspects, the drug delivery system can be configured to release on an average from about 0.001 to about 0.013 mg per day of ethinyl estradiol and on an average from about 0.10 to about 0.12 mg of etonogestrel per day for about 21 days after administration to the subject's vaginal tract. In some aspects, the drug delivery system can be configured to release on an average from about 0.001 to about 0.013 mg per day of ethinyl estradiol and on an average from about 0.10 to about 0.12 mg of etonogestrel per day for about 24 days after administration to the subject's vaginal tract. In some aspects, the drug delivery system can be configured to release on an average from about 0.001 to about 0.013 mg per day of ethinyl estradiol and on an average from about 0.10 to about 0.12 mg of etonogestrel per day for about 26 days after administration to the subject's vaginal tract. In some aspects, the drug delivery system can be configured to release on an average from about 0.001 to about 0.013 mg per day of ethinyl estradiol and on an average from about 0.10 to about 0.12 mg of etonogestrel per day for about 3 months after administration to the subject's vaginal tract. In some aspects, the drug delivery system can be configured to release on an average about 0.010 mg per day of ethinyl estradiol and on an average about 0.12 mg of etonogestrel per day for about 21 days after administration to the subject's vaginal tract. In some aspects, the drug delivery system can be configured to release on an average about 0.010 mg per day of ethinyl estradiol and on an average about 0.12 mg of etonogestrel per day for about 24 days after administration to the subject's vaginal tract. In some aspects, the drug delivery system can be configured to release on an average about 0.010 mg per day of ethinyl estradiol and on an average about 0.12 mg of etonogestrel per day for about 26 days after administration to the subject's vaginal tract. In some aspects, the drug delivery system can be configured to release on an average about 0.010 mg per day of ethinyl estradiol and on an average about 0.12 mg of etonogestrel per day for about 3 months after administration to the subject's vaginal tract. In some aspects, the drug delivery system can be configured to release less than 0.027 mg of ethinyl estradiol each day after insertion of the drug delivery device to a subject.

In some aspects, the drug delivery system can be configured to release less than a daily amount of 0.030, 0.029, 0.028, 0.027, 0.026, 0.025, or 0.024 mg (milligrams) of ethinyl estradiol during the burst release phase. In some aspects, the drug delivery system is configured to release less than a daily amount of 0.027 mg of ethinyl estradiol within the burst period after insertion of the drug delivery device to a subject. In some aspects, the burst period is the first 24 hr after insertion of the drug delivery device to a subject.

In some aspects, the core and the membrane are each independently made of a thermoplastic polymer or an elastomer. In some aspects, the core can be made of a material selected from the group consisting of low-density polyethylene, ethylene-vinyl acetate copolymers, thermoplastic polyurethane, styrene-butadiene-styrene copolymers, and a combination thereof. In some aspects, the membrane can be made of a material selected from the group consisting of olefins, vinyl polymers, rubber polymers, silicon polymers, microporous polymers, diffusion polymers, polyethylene, ethylene-vinyl acetate copolymers, thermoplastic polyurethanes, polyether-ester polymers, cellulose, and a combination thereof. In some aspects, the core can be made of an ethylene-vinyl acetate copolymer having a vinyl acetate relative molar content from 25 to 35%. In some aspects, core can be made of an ethylene-vinyl acetate copolymer having a vinyl acetate relative molar content of about 28%. In some aspects, the membrane can be made of an ethylene-vinyl acetate copolymer having a relative molar content from 8 to 10%. In some aspects, the membrane can be made of an ethylene-vinyl acetate copolymer having a relative molar content of about 9%. In some aspects, the core can comprise ethylene-vinyl acetate copolymer from about 81 wt. % to about 92 wt. % relative to the total weight of the drug delivery system. In some aspects, the core can comprise ethylene-vinyl acetate copolymer from about 85 wt. % to about 87 wt. % relative to the total weight of the drug delivery system. In some aspects, the membrane can comprise ethylene-vinyl acetate copolymer from about 11.7 wt. % to about 14.3 wt. % relative to the total weight of the drug delivery system. In some aspects, the membrane can comprise ethylene-vinyl acetate copolymer from about 12.5 wt. % to about 13.5 wt. % relative to the total weight of the drug delivery system.

In some aspects, the membrane further can comprise a plasticizer selected from fatty acids (and esters thereof), pyrrolidones, propylene glycol derivatives, glycerides, and a non-ionic surfactant.

In some aspects, the ring can have an outer diameter of from about 52 millimeters to about 54 millimeters. In some aspects, the ring can have an outer diameter of about 53.5 millimeters. In some aspects, the drug delivery system can have an inner diameter of from about 44 millimeters to about 47 millimeters. In some aspects, the drug delivery system can have an inner diameter of about 45.8 millimeters.

### BRIEF DESCRIPTION OF THE DRAWINGS

This description of the exemplary embodiments is intended to be read in connection with the accompanying drawings, which are to be considered part of the entire written description. The features of the embodiments described herein will be more fully disclosed in the following detailed description, which is to be considered together with the accompanying drawings wherein like numbers refer to like parts and further wherein. The drawing figures are not necessarily to scale and certain features may be shown exaggerated in scale or in somewhat schematic form in the interest of clarity and conciseness.

FIG. 1 shows a planar view of a drug delivery system (1) in the form of an annular vaginal ring having a ring-shaped core (12) surrounded on all sides by a membrane or a skin (11 and 13) where the membrane is permeable to active agents, such as contraceptive agents, included in the core and controls the rate of release of at least one active agent included in the core. One or more drugs or active agents may be evenly or unevenly dissolved, spread, distributed or included within the core of the ring. The vaginal ring is made of soft, resilient, pliable material such that it may be administered and retained in the vaginal tract of the subject.

FIG. 2 is a planar view of a drug delivery system (2) in the form of an annular vaginal ring having a ring-shaped core (23) surrounded on all sides by a membrane or a skin (21, 24) where the membrane controls the rate of release of at least one drug or active agent (22) included in the core. One or more active agents or drugs (22) are included in the core of the ring in the form of granules, crystals, pellets or other similar structures. The drug or the active agent may be distributed evenly or unevenly within the core of the ring. The vaginal ring is optionally made of soft, resilient, pliable material such that it may be administered and retained in the vaginal tract of the subject.

FIG. 3 is a drug delivery system (3) in the form of multiple annular rings where each ring has a core (31, 32, 33) and the core is optionally surrounded by a membrane or skin. Each of the rings may include the same active agent, a combination of active agents, or each ring may include a different active agent. The rings may be joined together, wrapped together, or concatenated together for easy delivery to a subject. Optionally, each ring may include a membrane or a skin where the membrane controls the rate of release of at least one active agent included within the core of the ring. In one embodiment, at least one ring is made of soft, resilient, pliable material such that it may be administered and retained in the vaginal tract of the subject.

FIG. 4 is a drug delivery system (4) in the form of a vaginal ring where the ring comprises a retaining ring (42) that is attached to multiple containers (41). The containers, for example, may include a drug or an active agent that is distributed evenly or unevenly within the containers. The container may, in one instance, include one or more drugs or active agents dissolved in a suitable solvent. The ring may optionally be surrounded on all sides by a membrane where the membrane controls the rate of release of at least one drug or active agent included within one or more containers.

FIG. 5 is a drug delivery system (5) in the form of an annular vaginal ring that comprises a core (51) where the core comprises two compartments (52, 53) embedded within the core. Each compartment comprises a drug or an active agent or a combination of drugs and active agents. The ring may optionally be surrounded on all sides by a membrane where the membrane controls the rate of release of at least one drug or active agent included within the compartment.

FIG. 6 is the drug release profile of etonogestrel from the reference product and a representative embodiment of the drug delivery system of this disclosure as described in Example 1.

FIG. 7 is the drug release profile of ethinyl estradiol from the reference product and a representative embodiment of the drug delivery system of this disclosure as described in Example 1.

### DETAILED DESCRIPTION

The invention is not limited to particular drug delivery systems, processes, compounds, or methodologies described, as these may vary. The terminology used in the description is for the purpose of describing particular versions or embodiments only, and is not intended to limit the scope of the invention. Unless defined otherwise, all technical and scientific terms used herein have the same meanings as commonly understood by one of ordinary skill in the art. Any methods and materials similar or equivalent to those described herein can be used in the practice or testing of embodiments of the invention, the preferred methods, devices, and materials described herein.

The active agent, drugs, or therapeutics of the invention include antifertility or contraceptive steroids. These steroids include progestogenic steroids that have antifertility properties and estrogenic steroids that also have antifertility properties. These active agents can be of naturally occurring or synthetic origin.

### Definitions

As referred to herein, the term “steroidal estrogenic compound” comprises, but not limited to, estrogen and compounds that imitate/mimic the effects of estrogen in a human or animal subject. They can either be synthetic or natural chemical compounds. Estrogen is the predominant sex hormone in women, affecting the development and function of the female reproductive system, including the endometrium, uterus and mammary glands. Estrogen is commonly used as a contraceptive, and is a major component of menopausal hormone therapy. Estrogens are also used in postmenopausal women to treat urinary stress incontinence, dizziness, fatigue, irritability, and to prevent osteoporosis. Endogenous natural estrogens are C18 steroids and include estrone (E1), estradiol (E2), estriol (E3), and estretrol (E4). Some examples of steroidal estrogenic compounds that may be used in the invention are α-estradiol; α-estradiol-3-benzoate; 17α-cyclopentanepropionate estradiol; 1,3,5,(10)-estratriene-3, 17α-diol dipropionate; estra-1,3,5(10)-triene-3, 17α-diol valerate; estrone; ethinyl estradiol; 17-ethinyl estradiol-3-methyl ether; mestranol, 17-ethinyl estradiol-3-cyclopentoether estroil; estradiol (E2); estriol (E3); and estretrol (E4) and mixtures thereof, and the like.

As referred to herein, the term “steroidal progestogenic compound” comprises, but not limited to, progestogens, progestins, and compounds that imitate the effects of progestin in a human or animal subject. Progestogens generically describe steroids possessing progestational activity and the term progestins are used to describe synthetic steroids that have progestational effects. Progesterone is an endogenous steroid and progestogen sex hormone involved in the menstrual cycle, pregnancy, and embryogenesis of humans and other species. It belongs to a group of steroid hormones called the progestogens, and is the major progestogen in the body. Progesterone has a variety of important functions in the body. It is also a crucial metabolic intermediate in the production of other endogenous steroids, including the sex hormones and the corticosteroids, and plays an important role in brain function as a neurosteroid.

As used herein, the singular forms “a”, “an” and “the” include plural reference unless the context clearly dictates otherwise.

As used herein, the term “about”, is intended to qualify the numerical values which it modifies, denoting such a value as variable within a margin of error. When no particular margin of error, such as a standard deviation to a mean value, is recited, the term “about” means plus or minus 10% of the numerical value of the number with which it is being used. Therefore, e.g., about 50% means in the range of 45%-55%.

As used herein, the terms “patient” and “subject” are interchangeable and may be taken to mean any living organism that may be treated with drug delivery systems, compounds or compositions of the invention. As such, the terms “patient” and “subject” may include, but are not limited to, any non-human mammal, primate or human. In some embodiments, the “patient” or “subject” is an adult, child, infant, or fetus. In some embodiments, the term “patient” or “subject” is a human. In some embodiments, the term “patient” or “subject” is a mammal, such as mice, rats, other rodents, rabbits, dogs, cats, swine, cattle, sheep, horses, primates, or humans.

In each of the embodiments disclosed herein, the active agents, therapeutics, pharmaceutical compositions, and methods may be utilized with or on a subject in need of such treatment, which may also be referred to as “in need thereof.” As used herein, the phrase “in need thereof” means that the subject has been identified as having a need for the particular method or treatment or that the treatment has been given to the subject for a particular purpose.

The terms “treat,” “treating,” and “treatment” are meant to include alleviating or abrogating a pregnancy, the chances of conception of an offspring, a disease/disorder, or one or more of the symptoms associated with a disease/disorder or alleviating or eradicating the cause(s) of the disease/disorder itself. The terms “treat,” “treating,” and “treatment” also include prevention, for example, prevention of a pregnancy. The terms “prevent,” “preventing,” and “prevention” refer to a method of delaying or precluding the onset of a disease/disorder; and/or its attendant symptoms, barring a subject from acquiring a disease/disorder or reducing a subject's risk of acquiring a disease/disorder; barring a subject from conceiving an offspring and/or preventing a pregnancy.

As used herein, the term “administering” when used in conjunction with a drug delivery system means to administer the delivery system directly or indirectly into or onto a target tissue or to a patient whereby the drug or therapeutic in the delivery system locally or systemically affects the tissue or the patient. “Administering” a drug delivery system or a composition may be accomplished by vaginal administration, oral administration, injection, infusion, inhalation, implantation, absorption or by any method in combination with other known techniques. “Administering” may include the act of self-administration or administration by another person such as by a health care provider.

As used herein, the term “pharmaceutical composition” means a composition including at least one or more active agents or compounds described in the invention. The pharmaceutical composition is amenable to investigation for a specified, efficacious outcome in a mammal (for example, without limitation, a human). Those of ordinary skill in the art will understand and appreciate the techniques appropriate for determining whether the active agents of the pharmaceutical composition have a desired efficacious outcome based upon the needs of the artisan.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004). Where relevant, all embodiments of the invention, including the drugs, active agents or compounds disclosed herein, may be formulated such that they include a “pharmaceutically acceptable carrier,” “pharmaceutically acceptable excipient,” “physiologically acceptable carrier,” or “physiologically acceptable excipient.”

The terms “therapeutically effective amount” or “therapeutic dose” are used interchangeably and refer to the amount of an active agent, pharmaceutical compound, or composition that elicits a clinical, biological or medicinal response in a tissue, system, animal, individual, or human that is being sought by a researcher, veterinarian, medical doctor, or other clinical professional. A clinical, biological or medical response may include, for example, one or more of the following: (1) preventing a pregnancy, disease, condition or disorder in an individual, (2) inhibiting a pregnancy, disease, condition or disorder in an individual, and (3) ameliorating a disease, condition or disorder in an individual that is experiencing or exhibiting the pathology or symptoms of the disease, condition or disorder or reversing the pathology and/or symptoms experience or exhibited by the individual.

The term “pharmaceutically acceptable salt” for the purpose of the invention is meant to indicate, without any limitation, those salts which are within the scope of sound medical judgment, suitable for use in contact with the tissues of a patient, without undue toxicity, irritation, allergic response and the like, and are commensurate with a reasonable benefit/risk ratio. Pharmaceutically acceptable salts suitable for the invention are well known in the art and described in, for instance, Berge et al., “Pharmaceutical Salts,” J. Pharm. Sci., 66(1):1-19 (1977).

The phrase “delivery system” as used herein refers to a system manufactured in the form of a delivery device, which provides a pre-programmed, unattended delivery of an agent, optionally, at a controlled rate, and for a period of time (or total period of time) established to meet a specific need.

The phrase “ultra-low dose” when used in the context of drug delivery systems described herein is considered to be those drug delivery systems that release less than 0.013 mg of steroid estrogenic compound per day. For example, an ultra-low dose can be about 0.010 mg per day of ethinyl estradiol or an average about 0.010 mg of ethinyl estradiol per day for about 21 days, about 24 days, about 26 days, or about 3 months.

The phrase “burst release” when used in the context of drug delivery systems described herein refers to the initial amount of drug released from the drug delivery system. The initial period can be measured within the first 1 hour, first 8 hours, or first 24 hours after introduction of the drug delivery system to a subject. In some embodiments, the burst release period is the first 24 hours after introduction of the drug delivery system to a subject. Some treatments such as wound healing require an initial burst release of a therapeutic agent followed by continuous gradually decreasing amounts of the therapeutic agent. However, other forms of treatment, such as contraceptive prevention, suffer from side effects due to a high burst release of the therapeutic agent but then require consistent about average dosage release amount of the therapeutic agent following the initial period. In some embodiments, the drug delivery system comprises ethinyl estradiol and the burst release amount of ethinyl estradiol is less than 0.030, 0.029, 0.028, 0.027, 0.026, 0.025, or 0.024 mg (milligrams). In some embodiments, the burst release amount of ethinyl estradiol is less than 0.030 mg. In some embodiments, the burst release amount of ethinyl estradiol is less than 0.029 mg. In some embodiments, the burst release amount of ethinyl estradiol is less than 0.028 mg. In some embodiments, the burst release amount of ethinyl estradiol is less than 0.027 mg. In some embodiments, the burst release amount of ethinyl estradiol is less than 0.026 mg. In some embodiments, the burst release amount of ethinyl estradiol is less than 0.025 mg. In some embodiments, the burst release amount of ethinyl estradiol is less than 0.024 mg. The burst release amounts can be measured in elution media using the methods described herein. In some embodiments, the serum concentration of ethinyl estradiol after the burst release period can range from 5-15 ng/L, as measured by HPLC-MS or HPLC-UV. In some embodiments, the serum concentration of ethinyl estradiol after the burst release period can range from 10-15 ng/L. Low serum concentrations of ethinyl estradiol distinguish the drug delivery systems and methods of their use as described herein from reference products which risk side-effects from higher ethinyl estradiol serum concentrations after administration of the product to a subject.

The phrase “sustained release” when used in the context of drug delivery systems described herein refers to the about constant, or slowly reducing, release of a therapeutic agent over an extended period of time after administration or introduction of the drug delivery system to a subject. The extended period of time is after the initial period of time, which can be within 1 hr, 8 hr, or 24 hr. The drug delivery systems described herein are designed to exhibit a sufficient sustained release dose of ethinyl estradiol to a subject such that the amount of ethinyl estradiol in the subject is above the minimum effective concentration.

The phrase “minimum effective concentration” or “MEC” when used in the context of drug delivery systems described herein refers to the lower level of a therapeutic agent in the body of a subject that will still be effective.

### Methods

Some embodiments of the invention are directed to drug delivery system for use in contraception. The drug delivery system is administered to a subject's vaginal tract to achieve contraception, and the system comprises a core comprising a steroidal estrogenic compound and a steroidal progestogenic compound. The core of the system is, optionally, covered by a membrane or a skin. The drug delivery system is such that when it is placed in a subject's vaginal tract, it delivers a therapeutically effective dose steroidal progestogenic compound and a therapeutically effective dose of the steroidal estrogenic compound to the subject at a controlled rate for a period of time to produce a contraceptive effect in the subject. In some embodiments, the therapeutically effective dose of steroidal estrogenic compound is an ultra-low dose.

In some embodiments, the drug delivery system of the invention causes simultaneous release of two or more active agent wherein the active agent can be, e.g., the steroidal estrogenic compound or the steroidal progestogenic compound. In an embodiment, the drug delivery system releases the active agents in a substantially constant ratio for a period of time (or total period of time). In some embodiments, the drug delivery system or the core of the drug delivery system comprises a fixed/constant ratio of the steroidal estrogenic compound to the steroidal progestogenic compound for a period of time (or total period of time). The above-mentioned substantially constant ratio and the fixed/constant ratio are examples of a controlled rate. The system may have one or multiple controlled rates for the period of time (or total period of time).

The steroidal estrogenic compound for the purposes of this invention can be selected form a variety of well-known synthetic and naturally occurring estrogenic compounds. For example, the steroidal estrogenic compound can be selected from the group consisting of ethinyl estradiol; estradiol; α-estradiol; α-estradiol-3-benzoate; 17α-cyclopentanepropionate estradiol; 1,3,5,(10)-estratriene-3, 17α-diol dipropionate; estra-1,3,5(10)-triene-3, 17α-diol valerate; 17-ethinyl estradiol-3-methyl ether; mestranol, 17-ethinyl estradiol-3-cyclopentoether estroil; estrone (E1); estradiol (E2); estriol (E3); or estretrol (E4) or mixtures thereof, and the like. In one preferred embodiment, the drug delivery systems of the invention include one or more bioidentical estrogens, such as estrone (E1), estradiol (E2), estriol (E3), or estretrol (E4).

In a preferred embodiment, the steroidal estrogenic compound is ethinyl estradiol. In another preferred embodiment, the steroidal estrogenic compound is estradiol.

The steroidal progestogenic compound for the purposes of this invention can be selected form a variety of well-known synthetic and naturally occurring progestogenic compounds. For example, the steroidal progestogenic compound can be selected from the group consisting of drospirenone, norethindrone, norethindrone acetate, levonorgestrel, etonogestrel, and norgestimate.

### Drug Delivery Systems

Disclosed herein is a drug delivery system in the form of intravaginal ring that delivers an ultra-low dose of at least one estrogenic compound to reduce side effects in females without affecting its efficacy.

The lower ethinyl estradiol concentration and exposure has a potential benefit of decreasing the risk of estrogen associated cancers including endometrial cancer and breast cancer as well as decreasing the risk of stroke, DVT (deep vein thrombosis), venous thromboembolism, pulmonary embolism and myocardial infarction. In contrast, etonogestrel when administered at conventional concentrations has no common serious side effects. Thus, the inventors recognized that a dual release drug delivery device with an ultra-low dose of ethinyl estradiol and a conventional dose of etonogestrel would reduce side effects of a contraceptive device comprising ethinyl estradiol.

Not to be bound by theory, but the inventors recognized that a drug delivery system comprising an ultra-low dose of ethinyl estradiol would reduce the burst effect of the drug delivery system for the time immediately following insertion of the drug delivery system to the subject. Conventional drug delivery systems comprising higher doses of ethinyl estradiol risk presenting a toxic- or side effect producing-dose level of ethinyl estradiol after insertion of the drug delivery device to a subject (Krishnan et al., Int J Womens Health. 2010; 2: 235-239; PMID: 21151728). The drug delivery devices of the present disclosure, however, avoid such a toxic- or side effect producing-dose level of ethinyl estradiol during the burst phase by reducing the amount of ethinyl estradiol in the drug delivery system, while retaining the ability to slowly release ethinyl estradiol during the indicated course of the menstrual cycle. The sustained release rate is about linear over time resulting a maintained plasma level EE concentration for contraceptive effect.

Some embodiments of the invention are directed to a drug delivery system having combination of progestin and ultra-low dose estrogen for the purpose of contraception. In some embodiments, the drug delivery system is such that the steroidal progestogenic compound is etonogestrel and the steroidal estrogenic compound is ethinyl estradiol. In some embodiments, the drug delivery system is such that the steroidal progestogenic compound is norethindrone acetate and the steroidal estrogenic compound is ethinyl estradiol. In some embodiments, the drug delivery system is such that the steroidal progestogenic compound is levonorgestrel and the steroidal estrogenic compound is ethinyl estradiol. In some embodiments, the drug delivery system is such that the steroidal progestogenic compound is drospirenone and the steroidal estrogenic compound is ethinyl estradiol.

In some embodiments, the drug delivery system comprises ethinyl estradiol and the system delivers about 0.010 mg per day of ethinyl estradiol to the subject for about 21 days after administration. In some embodiments, the drug delivery system comprises ethinyl estradiol and the system delivers about 0.010 mg per day of ethinyl estradiol to the subject for about 24 days after administration. In some embodiments, the drug delivery system comprises ethinyl estradiol and the system delivers about 0.010 mg per day of ethinyl estradiol to the subject for about 26 days after administration. In some embodiments, the drug delivery system comprises ethinyl estradiol and the system delivers about 0.010 mg per day of ethinyl estradiol to the subject for about 3 months after administration.

The drug delivery systems described herein release the steroidal progestogenic compound, steroidal estrogenic compound, or a combination thereof for a period of time to the subject. In some embodiments, the drug delivery system delivers a therapeutically effective dose of the steroidal progestogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days, or any time point between the aforementioned values, to the subject. In some embodiments, the drug delivery system delivers a therapeutically effective dose of the steroidal estrogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days, or any time point between the aforementioned values, to the subject. In some embodiments, the drug delivery system delivers a therapeutically effective dose of the steroidal estrogenic compound and a therapeutically effective dose of the steroidal progestogenic compound for a period of time wherein the time is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 15 days, about 20 days, about 21 days, about 24 days, about 25 days, about 26 days, about 30 days, about 35 days, about 40 days, about 45 days, about 50 days, about 55 days, about 60 days, about 65 days, about 70 days, about 75 days, about 80 days, about 85 days, or about 90 days, or any time point between the aforementioned values, to the subject. In a preferred embodiment, the drug delivery system delivers a therapeutically effective dose of the steroidal progestogenic compound and the steroidal estrogenic compound for a period of about or at least 21 days or up to 3 months to the subject's vaginal tract.

In some embodiments, the drug delivery system comprises from about 1 mg etonogestrel to about 75 mg etonogestrel. In some embodiments, the drug delivery system comprises about 5, 10, 15, 20, 25, 30, 35, 40, 45, 50, 55, 60, 65, 70, or 75 mg, or any value between the aforementioned values, of etonogestrel. In some embodiments, the drug delivery system comprises from about 0.1 mg to about 7.5 mg ethinyl estradiol. In some embodiments, the drug delivery system comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg, or any value between the aforementioned values, of ethinyl estradiol. In a preferred embodiment, the drug delivery system comprises about 11.7 mg of etonogestrel and about 1.8 mg of ethinyl estradiol. In another preferred embodiment, the drug delivery system comprises about 13.4 mg of etonogestrel and about 2.1 mg of ethinyl estradiol. In another preferred embodiment, the drug delivery system comprises about 14.5 mg of etonogestrel and about 2.2 mg of ethinyl estradiol. In another preferred embodiment, the drug delivery system comprises about 35.1 mg of etonogestrel and about 5.4 mg of ethinyl estradiol. In some embodiments, the drug delivery system releases on an average from about 0.1 mg to about 0.2 mg of etonogestrel per day for about 21 days. In some embodiments, the drug delivery system releases on an average from about 0.1 mg to about 0.2 mg of etonogestrel per day for about 24 days. In some embodiments, the drug delivery system releases on an average from about 0.1 mg to about 0.2 mg of etonogestrel per day for about 26 days. In some embodiments, the drug delivery system releases on an average from about 0.1 mg to about 0.2 mg of etonogestrel per day for about 3 months. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 21 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 24 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 26 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 3 months after administration to the subject's vaginal tract. In a preferred embodiment, the system releases on an average about 0.12 mg of etonogestrel per for day for about 21 days after administration to the subject's vaginal tract. In another preferred embodiment, the system releases on an average about 0.12 mg of etonogestrel per for day for about 24 days after administration to the subject's vaginal tract. In another preferred embodiment, the system releases on an average about 0.12 mg of etonogestrel per for day for about 26 days after administration to the subject's vaginal tract. In another preferred embodiment, the system releases on an average about 0.12 mg of etonogestrel per for day for about 3 months after administration to the subject's vaginal tract. In some embodiments, the drug delivery system releases on an average from about 0.001 to about 0.013 mg of ethinyl estradiol per day for about 21 days after administration to the subject's vaginal tract. In some embodiments, the drug delivery system releases on an average from about 0.001 to about 0.013 mg of ethinyl estradiol per day for about 24 days after administration to the subject's vaginal tract. In some embodiments, the drug delivery system releases on an average from about 0.001 to about 0.013 mg of ethinyl estradiol per day for about 26 days after administration to the subject's vaginal tract. In some embodiments, the drug delivery system releases on an average from about 0.001 to about 0.013 mg of ethinyl estradiol per day for about 3 months after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 21 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 24 days after administration to the subject's vaginal tract. In some embodiments, the drug delivery system releases on an average about 0.001, 0.002, 0.003, 0.004, 0.005, 0.006, 0.07, 0.008, 0.009, 0.010, 0.012, and 0.013 mg, or any value between the aforementioned values, of ethinyl estradiol per for day for about 26 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In some embodiments, the steroidal progestogenic compound in the drug delivery system is norethindrone acetate and the steroidal estrogenic compound in the drug delivery system is ethinyl estradiol. Such drug delivery system comprises from about 50 mg to about 750 mg of norethindrone acetate. In some embodiments, the drug delivery system comprises about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 mg, or any value between the aforementioned values, of norethindrone acetate. This drug delivery system comprises from about 0.1 mg to about 7.5 mg ethinyl estradiol. In some embodiments, the drug delivery system comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg, or any value between the aforementioned values, of ethinyl estradiol. In a preferred embodiment, the drug delivery system comprises about 100 mg of norethindrone acetate and about 1.8 mg of ethinyl estradiol. In another preferred embodiment, the drug delivery system comprises about 114 mg of norethindrone acetate and about 2.1 mg of ethinyl estradiol. In another preferred embodiment, the drug delivery system comprises about 124 mg of norethindrone acetate and about 2.2 mg of ethinyl estradiol. In another preferred embodiment, the drug delivery system comprises about 300 mg of norethindrone acetate and about 5.4 mg of ethinyl estradiol. In some embodiments, the drug delivery system releases on an average from about 0.1 mg to about 2 mg of norethindrone acetate per day for about 21 days. In some embodiments, the drug delivery system releases on an average from about 0.1 mg to about 2 mg of norethindrone acetate per day for about 24 days. In some embodiments, the drug delivery system releases on an average from about 0.1 mg to about 2 mg of norethindrone acetate per day for about 26 days. In some embodiments, the drug delivery system releases on an average from about 0.1 mg to about 2 mg of norethindrone acetate per day for about 3 months. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 21 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2.0 mg, or any value between the aforementioned values, of norethindrone acetate per for day for about 24 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 26 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 3 months after administration to the subject's vaginal tract. In a preferred embodiment, the system releases on an average about 1 mg of norethindrone acetate per for day for about 21 days after administration to the subject's vaginal tract. In another preferred embodiment, the system releases on an average about 1 mg of norethindrone acetate per for day for about 24 days after administration to the subject's vaginal tract. In another preferred embodiment, the system releases on an average about 1 mg of norethindrone acetate per for day for about 26 days after administration to the subject's vaginal tract. In another preferred embodiment, the system releases on an average about 1 mg of norethindrone acetate per for day for about 3 months after administration to the subject's vaginal tract. In some embodiments, the drug delivery system releases on an average from about 0.001 to about 0.013 mg of ethinyl estradiol per day for about 21 days after administration to the subject's vaginal tract. In some embodiments, the drug delivery system releases on an average from about 0.001 to about 0.013 mg of ethinyl estradiol per day for about 24 days after administration to the subject's vaginal tract. In some embodiments, the drug delivery system releases on an average from about 0.001 to about 0.013 mg of ethinyl estradiol per day for about 26 days after administration to the subject's vaginal tract. In some embodiments, the drug delivery system releases on an average from about 0.001 to about 0.013 mg of ethinyl estradiol per day for about 3 months after administration to the subject's vaginal tract. In some embodiments, the drug delivery system releases on an average about 0.001, 0.002, 0.003, 0.004, 0.005, 0.006, 0.007, 0.08, 0.009, 0.010, 0.012, and 0.013 mg, or any value between the aforementioned values, of ethinyl estradiol per for day for about 21 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 24 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 26 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In some embodiments, the drug delivery system comprises from about 4 mg levonorgestrel to about 300 mg levonorgestrel. In some embodiments, the drug delivery system comprises about 4, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mg, or any value between the aforementioned values, of levonorgestrel. In some embodiments, the drug delivery system comprises from about 0.1 mg to about 7.5 mg ethinyl estradiol. In some embodiments, the drug delivery system comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg, or any value between the aforementioned values, of ethinyl estradiol. In a preferred embodiment, the drug delivery system comprises about 49 mg of levonorgestrel and about 1.8 mg of ethinyl estradiol. In another preferred embodiment, the drug delivery system comprises about 56 mg of levonorgestrel and about 2.1 mg of ethinyl estradiol. In another preferred embodiment, the drug delivery system comprises about 60 mg of levonorgestrel and about 2.2 mg of ethinyl estradiol. In another preferred embodiment, the drug delivery system comprises about 146 mg of levonorgestrel and about 5.4 mg of ethinyl estradiol. In some embodiments, the drug delivery system releases on an average from about 0.45 mg to about 0.55 mg of levonorgestrel per day for about 21 days. In some embodiments, the drug delivery system releases on an average from about 0.45 mg to about 0.55 mg of levonorgestrel per day for about 24 days. In some embodiments, the drug delivery system releases on an average from about 0.45 mg to about 0.55 mg of levonorgestrel per day for about 26 days. In some embodiments, the drug delivery system releases on an average from about 0.45 mg to about 0.55 mg of levonorgestrel per day for about 3 months. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 21 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 24 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 26 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 3 months after administration to the subject's vaginal tract. In a preferred embodiment, the system releases on an average about 0.50 mg of levonorgestrel per for day for about 21 days after administration to the subject's vaginal tract. In another preferred embodiment, the system releases on an average about 0.50 mg of levonorgestrel per for day for about 24 days after administration to the subject's vaginal tract. In a preferred embodiment, the system releases on an average about 0.50 mg of levonorgestrel per for day for about 26 days after administration to the subject's vaginal tract. In another preferred embodiment, the system releases on an average about 0.50 mg of levonorgestrel per for day for about 3 months after administration to the subject's vaginal tract. In some embodiments, the drug delivery system releases on an average from about 0.001 to about 0.013 mg of ethinyl estradiol per day for about 21 days after administration to the subject's vaginal tract. In some embodiments, the drug delivery system releases on an average from about 0.001 to about 0.013 mg of ethinyl estradiol per day for about 24 days after administration to the subject's vaginal tract. In some embodiments, the drug delivery system releases on an average from about 0.001 to about 0.013 mg of ethinyl estradiol per day for about 26 days after administration to the subject's vaginal tract. In some embodiments, the drug delivery system releases on an average from about 0.001 to about 0.013 mg of ethinyl estradiol per day for about 3 months after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 21 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 24 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 26 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 3 months after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract. In another preferred embodiment, the system releases on an average about 0.010 mg of ethinyl estradiol per for day for about 3 months after administration to the subject's vaginal tract.

In some embodiments, the steroidal progestogenic compound in the drug delivery system is drospirenone and the steroidal estrogenic compound in the drug delivery system is ethinyl estradiol. Such drug delivery system comprises from about 150 mg to about 190 mg of drosprinone. In some embodiments, the drug delivery system comprises about 150, 160, 170, 180, or 190 mg, or any value between the aforementioned values, of drospirenone. This drug delivery system comprises from about 0.1 mg to about 2.5 mg ethinyl estradiol. In some embodiments, the drug delivery system comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, or 2.5 mg, or any value between the aforementioned values, of ethinyl estradiol. In a preferred embodiment, the drug delivery system comprises about 300 mg of drospirenone and about 1.8 mg of ethinyl estradiol. In another preferred embodiment, the drug delivery system comprises about 343 mg of drospirenone and about 2.1 mg of ethinyl estradiol. In another preferred embodiment, the drug delivery system comprises about 371 mg of drospirenone and about 2.2 mg of ethinyl estradiol. In some embodiments, the drug delivery system releases on an average from about 0.1 mg to about 4 mg of drospirenone per day for about 21 days. In some embodiments, the drug delivery system releases on an average from about 0.1 mg to about 4 mg of drospirenone per day for about 24 days. In some embodiments, the drug delivery system releases on an average from about 0.1 mg to about 4 mg of drospirenone per day for about 26 days. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 21 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 24 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 26 days after administration to the subject's vaginal tract. In a preferred embodiment, the system releases on an average about 3 mg of drospirenone per for day for about 21 days after administration to the subject's vaginal tract. In another preferred embodiment, the system releases on an average about 3 mg of drospirenone per for day for about 24 days after administration to the subject's vaginal tract. In another preferred embodiment, the system releases on an average about 1 mg of drospirenone per for day for about 26 days after administration to the subject's vaginal tract. In some embodiments, the drug delivery system releases on an average from about 0.001 to about 0.013 mg of ethinyl estradiol per day for about 21 days after administration to the subject's vaginal tract. In some embodiments, the drug delivery system releases on an average from about 0.001 to about 0.013 mg of ethinyl estradiol per day for about 24 days after administration to the subject's vaginal tract. In some embodiments, the drug delivery system releases on an average from about 0.001 to about 0.013 mg of ethinyl estradiol per day for about 26 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 21 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 24 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.012, and 0.013 mg, or any value between the aforementioned values, of ethinyl estradiol per for day for about 26 days after administration to the subject's vaginal tract. In a preferred embodiment, the system releases on an average about 0.010 mg of ethinyl estradiol per for day for about 21 days after administration to the subject's vaginal tract. In another preferred embodiment, the system releases on an average about 0.010 mg of ethinyl estradiol per for day for about 24 days after administration to the subject's vaginal tract. In another preferred embodiment, the system releases on an average about 0.010 mg of ethinyl estradiol per for day for about 26 days after administration to the subject's vaginal tract.

In some embodiments, the steroidal progestogenic compound in the drug delivery system is etonogestrel and the steroidal estrogenic compound in the drug delivery system is estradiol. Such drug delivery system comprises from about 1 mg etonogestrel to about 75 mg etonogestrel. In some embodiments, the drug delivery system comprises about 5, 10, 15, 20, 25, 30, 35, 40, 45, 50, 55, 60, 65, 70, or 75 mg, or between any of the aforementioned values, of etonogestrel. In some embodiments, the drug delivery system comprises from about 10 mg to about 105 mg of estradiol. In some embodiments, the drug delivery system comprises about 10, 15, 20, 25, 30, 35, 40, 45, 50, 55, 60, 65, 70, 75, 80, 85, 90, 95, 100, or 105 mg, or any value between the aforementioned values, of estradiol. In a preferred embodiment, the drug delivery system comprises about 11.7 mg of etonogestrel and about 27 mg of estradiol. In another preferred embodiment, the drug delivery system comprises about 35.1 mg of etonogestrel and about 81 mg of estradiol. In some embodiments, the drug delivery system releases on an average from about 0.1 mg to about 0.2 mg of etonogestrel per day for about 21 days. In some embodiments, the drug delivery system releases on an average from about 0.1 mg to about 0.2 mg of etonogestrel per day for about 24 days. In some embodiments, the drug delivery system releases on an average from about 0.1 mg to about 0.2 mg of etonogestrel per day for about 26 days. In some embodiments, the drug delivery system releases on an average from about 0.1 mg to about 0.2 mg of etonogestrel per day for about 3 months. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 21 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 24 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 26 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per for day for about 3 months after administration to the subject's vaginal tract. In a preferred embodiment, the system releases on an average about 0.12 mg of etonogestrel per for day for about 21 days after administration to the subject's vaginal tract. In another preferred embodiment, the system releases on an average about 0.12 mg of etonogestrel per for day for about 24 days after administration to the subject's vaginal tract. In another preferred embodiment, the system releases on an average about 0.12 mg of etonogestrel per for day for about 26 days after administration to the subject's vaginal tract. In another preferred embodiment, the system releases on an average about 0.12 mg of etonogestrel per for day for about 3 months after administration to the subject's vaginal tract. In some embodiments, the drug delivery system releases on an average from about 0.05 to about 0.25 mg of estradiol per day for about 21 days after administration to the subject's vaginal tract. In some embodiments, the drug delivery system releases on an average from about 0.05 to about 0.25 mg of estradiol per day for about 24 days after administration to the subject's vaginal tract. In some embodiments, the drug delivery system releases on an average from about 0.05 to about 0.25 mg of estradiol per day for about 26 days after administration to the subject's vaginal tract. In some embodiments, the drug delivery system releases on an average from about 0.05 to about 0.25 mg of estradiol per day for about 3 months after administration to the subject's vaginal tract. In some embodiments, the drug delivery system releases on an average about 0.05, 0.1, 0.15, 0.2, or 0.25 mg, or any value between the aforementioned values, of estradiol per for day for about 21 days after administration to the subject's vaginal tract. In some embodiments, the drug delivery system releases on an average about 0.05, 0.1, 0.15, 0.2, or 0.25 mg, or any value between the aforementioned values, of estradiol per for day for about 24 days after administration to the subject's vaginal tract. In some embodiments, the drug delivery system releases on an average about 0.05, 0.1, 0.15, 0.2, or 0.25 mg, or any value between the aforementioned values, of estradiol per for day for about 26 days after administration to the subject's vaginal tract. In some embodiments, the drug delivery system releases on an average about 0.05, 0.1, 0.15, 0.2, or 0.25 mg, or any value between the aforementioned values, of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In some embodiments, the steroidal progestogenic compound in the drug delivery system is norethindrone acetate and the steroidal estrogenic compound in the drug delivery system is estradiol. Such drug delivery system comprises from about 50 mg to about 750 mg of norethindrone acetate. In some embodiments, the drug delivery system comprises about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, or 750 mg, or any value between the aforementioned values, of norethindrone acetate. In some embodiments, the drug delivery system comprises from about 10 mg to about 105 mg of estradiol. In some embodiments, the drug delivery system comprises about 10, 15, 20, 25, 30, 35, 40, 45, 50, 55, 60, 65, 70, 75, 80, 85, 90, 95, 100, or 105 mg, or any value between the aforementioned values, of estradiol. In a preferred embodiment, the drug delivery system comprises about 100 mg of norethindrone acetate and about 27 mg of estradiol. In another preferred embodiment, the drug delivery system comprises about 300 mg of norethindrone acetate and about 81 mg of estradiol. In some embodiments, the drug delivery system releases on an average from about 0.1 mg to about 2 mg of norethindrone acetate per day for about 21 days. In some embodiments, the drug delivery system releases on an average from about 0.1 mg to about 2 mg of norethindrone acetate per day for about 24 days. In some embodiments, the drug delivery system releases on an average from about 0.1 mg to about 2 mg of norethindrone acetate per day for about 26 days. In some embodiments, the drug delivery system releases on an average from about 0.1 mg to about 2 mg of norethindrone acetate per day for about 3 months. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 21 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 24 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 26 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg, or any value between the aforementioned values, of norethindrone acetate per for day for about 3 months after administration to the subject's vaginal tract. In a preferred embodiment, the system releases on an average about 1 mg of norethindrone acetate per for day for about 21 days after administration to the subject's vaginal tract. In another preferred embodiment, the system releases on an average about 1 mg of norethindrone acetate per for day for about 24 days after administration to the subject's vaginal tract. In another preferred embodiment, the system releases on an average about 1 mg of norethindrone acetate per for day for about 26 days after administration to the subject's vaginal tract. In another preferred embodiment, the system releases on an average about 1 mg of norethindrone acetate per for day for about 3 months after administration to the subject's vaginal tract. In some embodiments, the drug delivery system releases on an average from about 0.05 to about 0.25 mg of estradiol per day for about 21 days after administration to the subject's vaginal tract. In some embodiments, the drug delivery system releases on an average from about 0.05 to about 0.25 mg of estradiol per day for about 24 days after administration to the subject's vaginal tract. In some embodiments, the drug delivery system releases on an average from about 0.05 to about 0.25 mg of estradiol per day for about 26 days after administration to the subject's vaginal tract. In some embodiments, the drug delivery system releases on an average from about 0.05 to about 0.25 mg of estradiol per day for about 3 months after administration to the subject's vaginal tract. In some embodiments, the drug delivery system releases on an average about 0.05, 0.1, 0.15, 0.2, or 0.25 mg, or any value between the aforementioned values, of estradiol per for day for about 21 days after administration to the subject's vaginal tract. In some embodiments, the drug delivery system releases on an average about 0.05, 0.1, 0.15, 0.2, or 0.25 mg, or any value between the aforementioned values, of estradiol per for day for about 24 days after administration to the subject's vaginal tract. In some embodiments, the drug delivery system releases on an average about 0.05, 0.1, 0.15, 0.2, or 0.25 mg, or any value between the aforementioned values, of estradiol per for day for about 26 days after administration to the subject's vaginal tract. In some embodiments, the drug delivery system releases on an average about 0.05, 0.1, 0.15, 0.2, or 0.25 mg, or any value between the aforementioned values, of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In some embodiments, the drug delivery system comprises from about 4 mg levonorgestrel to about 300 mg levonorgestrel. In some embodiments, the drug delivery system comprises about 4, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mg, or any value between the aforementioned values, of levonorgestrel. In some embodiments, the drug delivery system comprises from about 10 mg to about 105 mg of estradiol. In some embodiments, the drug delivery system comprises about 10, 15, 20, 25, 30, 35, 40, 45, 50, 55, 60, 65, 70, 75, 80, 85, 90, 95, 100, or 105 mg, or any value between the aforementioned values, of estradiol. In a preferred embodiment, the drug delivery system comprises about 49 mg of levonorgestrel and about 27 mg of estradiol. In another preferred embodiment, the drug delivery system comprises about 56 mg of levonorgestrel and about 31 mg of estradiol. In another preferred embodiment, the drug delivery system comprises about 60 mg of levonorgestrel and about 33 mg of estradiol. In another preferred embodiment, the drug delivery system comprises about 146 mg of levonorgestrel and about 81 mg of estradiol. In some embodiments, the drug delivery system releases on an average from about 0.45 mg to about 0.55 mg of levonorgestrel per day for about 21 days. In some embodiments, the drug delivery system releases on an average from about 0.45 mg to about 0.55 mg of levonorgestrel per day for about 24 days. In some embodiments, the drug delivery system releases on an average from about 0.45 mg to about 0.55 mg of levonorgestrel per day for about 26 days. In some embodiments, the drug delivery system releases on an average from about 0.45 mg to about 0.55 mg of levonorgestrel per day for about 3 months. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 21 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 24 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 26 days after administration to the subject's vaginal tract. In some embodiments, the drug delivery system releases on an average about 0.45, 0.46, 0.47, 0.48, 0.49, 0.50, 0.51, 0.52, 0.53, 0.54 or 0.55 mg, or any value between the aforementioned values, of levonorgestrel per for day for about 3 months after administration to the subject's vaginal tract. In a preferred embodiment, the system releases on an average about 0.50 mg of levonorgestrel per for day for about 21 days after administration to the subject's vaginal tract. In another preferred embodiment, the system releases on an average about 0.50 mg of levonorgestrel per for day for about 24 days after administration to the subject's vaginal tract. In another preferred embodiment, the system releases on an average about 0.50 mg of levonorgestrel per for day for about 26 days after administration to the subject's vaginal tract. In another preferred embodiment, the system releases on an average about 0.50 mg of levonorgestrel per for day for about 3 months after administration to the subject's vaginal tract. In some embodiments, the drug delivery system releases on an average from about 0.05 to about 0.25 mg of estradiol per day for about 21 days after administration to the subject's vaginal tract. In some embodiments, the drug delivery system releases on an average from about 0.05 to about 0.25 mg of estradiol per day for about 24 days after administration to the subject's vaginal tract. In some embodiments, the drug delivery system releases on an average from about 0.05 to about 0.25 mg of estradiol per day for about 26 days after administration to the subject's vaginal tract. In some embodiments, the drug delivery system releases on an average from about 0.05 to about 0.25 mg of estradiol per day for about 3 months after administration to the subject's vaginal tract. In some embodiments, the drug delivery system releases on an average about 0.05, 0.1, 0.15, 0.2, or 0.25 mg of estradiol per for day for about 21 days after administration to the subject's vaginal tract. In some embodiments, the drug delivery system releases on an average about 0.05, 0.1, 0.15, 0.2, or 0.25 mg, or any value between the aforementioned values, of estradiol per for day for about 24 days after administration to the subject's vaginal tract. In some embodiments, the drug delivery system releases on an average about 0.05, 0.1, 0.15, 0.2, or 0.25 mg, or any value between the aforementioned values, of estradiol per for day for about 26 days after administration to the subject's vaginal tract. In some embodiments, the drug delivery system releases on an average about 0.05, 0.1, 0.15, 0.2, or 0.25 mg, or any value between the aforementioned values, of estradiol per for day for about 3 months after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract. In another preferred embodiment, the system releases on an average about 0.15 mg of estradiol per for day for about 3 months after administration to the subject's vaginal tract.

In some embodiments, the steroidal progestogenic compound in the drug delivery system is drospirenone and the steroidal estrogenic compound in the drug delivery system is estradiol. Such drug delivery system comprises from about 150 mg to about 190 mg of drosprinone. In some embodiments, the drug delivery system comprises about 150, 160, 170, 180, or 190 mg, or any value between the aforementioned values, of drospirenone. In some embodiments, the drug delivery system comprises from about 10 mg to about 35 mg of estradiol. In some embodiments, the drug delivery system comprises about 10, 15, 20, 25, 30, or 35 mg, or any value between the aforementioned values, of estradiol. In a preferred embodiment, the drug delivery system comprises about 300 mg of drospirenone and about 27 mg of estradiol. In another preferred embodiment, the drug delivery system comprises about 343 mg of drospirenone and about 31 mg of estradiol. In another preferred embodiment, the drug delivery system comprises about 371 mg of drospirenone and about 33 mg of estradiol. In some embodiments, the drug delivery system releases on an average from about 0.1 mg to about 4 mg of drospirenone per day for about 21 days. In some embodiments, the drug delivery system releases on an average from about 0.1 mg to about 4 mg of drospirenone per day for about 24 days. In some embodiments, the drug delivery system releases on an average from about 0.1 mg to about 4 mg of drospirenone per day for about 26 days. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 21 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 24 days after administration to the subject's vaginal tract. In some embodiments, the drug delivery system releases on an average about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg, or any value between the aforementioned values, of drospirenone per for day for about 26 days after administration to the subject's vaginal tract. In a preferred embodiment, the system releases on an average about 3 mg of drospirenone per for day for about 21 days after administration to the subject's vaginal tract. In another preferred embodiment, the system releases on an average about 3 mg of drospirenone per for day for about 24 days after administration to the subject's vaginal tract. In another preferred embodiment, the system releases on an average about 1 mg of drospirenone per for day for about 26 days after administration to the subject's vaginal tract. In some embodiments, the drug delivery system releases on an average from about 0.05 to about 0.25 mg of estradiol per day for about 21 days after administration to the subject's vaginal tract. In some embodiments, the drug delivery system releases on an average from about 0.05 to about 0.25 mg of estradiol per day for about 24 days after administration to the subject's vaginal tract. In some embodiments, the drug delivery system releases on an average from about 0.05 to about 0.25 mg of estradiol per day for about 26 days after administration to the subject's vaginal tract. In some embodiments, the drug delivery system releases on an average about 0.05, 0.1, 0.15, 0.2, or 0.25 mg, or any value between the aforementioned values, of estradiol per for day for about 21 days after administration to the subject's vaginal tract. In some embodiments, the drug delivery system releases on an average about 0.05, 0.1, 0.15, 0.2, or 0.25 mg, or any value between the aforementioned values, of estradiol per for day for about 24 days after administration to the subject's vaginal tract. In some embodiments, the drug delivery system releases on an average about 0.05, 0.1, 0.15, 0.2, or 0.25 mg, or any value between the aforementioned values, of estradiol per for day for about 26 days after administration to the subject's vaginal tract. In a preferred embodiment, the system releases on an average about 0.15 mg of estradiol per for day for about 21 days after administration to the subject's vaginal tract. In another preferred embodiment, the system releases on an average about 0.15 mg of estradiol per for day for about 24 days after administration to the subject's vaginal tract. In another preferred embodiment, the system releases on an average about 0.15 mg of estradiol per for day for about 26 days after administration to the subject's vaginal tract.

The drug delivery system as disclosed herein can have different shapes or forms, such as, an implant, a sphere, a cylinder, an intrauterine system, a helical coil, a toroid, a spring, or a ring-shaped vaginal drug delivery system. When the drug delivery system is for intravaginal use it may be used for female medical indications, such as, for contraception.

In some embodiments the drug delivery system, e.g., the vaginal ring, has multiple sections and the active agents of the invention are present in different sections present within the ring. For instance, in an embodiment, the steroidal estrogenic compound and a steroidal progestogenic compound are enclosed in temporary or permanent separate sections of the core. In some embodiments, the steroidal estrogenic compound and a steroidal progestogenic compound are enclosed in the same section of the core. In some embodiments, the steroidal estrogenic compound and a steroidal progestogenic compound are enclosed in same and/or temporary or permanent separate compartments within the core.

In some embodiments, the invention is directed to a drug delivery system having combination of progestin and ultra-low dose estrogen for the purpose of contraception. In some embodiments, the drug delivery system is such that the steroidal progestogenic compound is etonogestrel and the steroidal estrogenic compound is ethinyl estradiol.

The lower ethinyl estradiol concentration and exposure has a potential benefit of decreasing the risk of estrogen associated cancers including endometrial cancer and breast cancer as well as decreasing the risk of stroke, DVT (deep vein thrombosis), venous thromboembolism, pulmonary embolism and myocardial infarction. In contrast, etonogestrel when administered at conventional concentrations has no common serious side effects. Thus, the inventors recognized that a dual release drug delivery device with an ultra-low dose of ethinyl estradiol and a conventional dose of etonogestrel would reduce side effects of a contraceptive device comprising ethinyl estradiol.

Not to be bound by theory, but the inventors recognized that a drug delivery system comprising an ultra-low dose of ethinyl estradiol would reduce the burst effect of the drug delivery system for the time immediately following insertion of the drug delivery system to the subject. Conventional drug delivery systems comprising higher doses of ethinyl estradiol risk presenting a toxic- or side effect producing-dose level of ethinyl estradiol after insertion of the drug delivery device to a subject. The drug delivery devices of the present disclosure, however, avoid such a toxic- or side effect producing-dose level of ethinyl estradiol by reducing the amount of ethinyl estradiol in the drug delivery system, while retaining the ability to slowly release ethinyl estradiol during the indicated course of the menstrual cycle.

In some embodiments, the therapeutically effective dose of ethinyl estradiol compound is an ultra-low dose of ethinyl estradiol. An ultra-low dose of ethinyl estradiol is 13 micrograms per day of ethinyl estradiol or less.

In some embodiments, the drug delivery system releases on an average from about 0.001 to about 0.013 mg of ethinyl estradiol per day, after the first day of insertion of the drug delivery device to a subject's vaginal tract. In some embodiments, the drug delivery system releases on an average from about 0.001 to about 0.010 mg of ethinyl estradiol per day, after the first day of insertion of the drug delivery device to a subject's vaginal tract.

In some embodiments, the drug delivery system releases on an average about 0.001, 0.002, 0.003, 0.004, 0.005, 0.006, 0.007, 0.008, 0.009, 0.010, 0.011, 0.012, and 0.013 mg of ethinyl estradiol per day, or any value between the aforementioned values, after the first day of insertion of the drug delivery device to a subject's vaginal tract.

In some embodiments, the drug delivery system releases less than 0.013 mg per day of ethinyl estradiol, after the first day of insertion of the drug delivery device to a subject's vaginal tract. In some embodiments, the drug delivery system releases on an average about 0.013 mg per day of ethinyl estradiol. In some embodiments, the drug delivery system releases on an average about 0.010 mg per day of ethinyl estradiol.

In some embodiments, the drug delivery system releases on an average from about 0.1 mg to about 0.2 mg of etonogestrel per day, after the first day of insertion of the drug delivery device to a subject's vaginal tract. In some embodiments, the drug delivery system releases on an average from about 0.1 mg to about 0.15 mg of etonogestrel per day. In some embodiments, the drug delivery system releases on an average from about 0.1 mg to about 0.12 mg of etonogestrel per day.

In some embodiments, the drug delivery system releases on an average about 0.10, 0.11, 0.12, 0.13, 0.14, 0.15, 0.16, 0.17, 0.18, 0.19 or 0.20 mg of etonogestrel per day, or any value between the aforementioned values, after the first day of insertion of the drug delivery device to a subject's vaginal tract.

In some embodiments, the drug delivery system releases about 0.12 mg of etonogestrel per day, after the first day of insertion of the drug delivery device to a subject's vaginal tract.

In some embodiments, the drug delivery system releases on an average about from about 0.001 to about 0.013 mg of ethinyl estradiol and about 0.1 mg to about 0.2 mg of etonogestrel per day. In some embodiments, the drug delivery system releases on an average about 0.010 mg of ethinyl estradiol and about 0.12 mg of etonogestrel per day.

The time duration over which the average amount of drug is released can be selected from day 0 to within the first 6 hours, 8 hours, 12 hours, or 24 hours after administration of the drug delivery system to a subject. In some embodiments, the time duration over which the average amount of drug is released can be after the initial burst phase of drug release. The time duration over which the average amount of drug is released can be selected from the 24^{th} to 48^{th} hour (end of day 1 to end of day 2), end of day 2 to end of day 3, end of day 3 to end of day 4, end of day 4 to end of day 5, end of day 5 to end of day 6, end of day 6 to end of day 7, end of day 7 to end of day 8, end of day 8 to end of day 9, end of day 9 to end of day 10, end of day 10 to end of day 11, end of day 11 to end of day 12, end of day 12 to end of day 13, end of day 13 to end of day 14, end of day 14 to end of day 15, end of day 15 to end of day 16, end of day 16 to end of day 17, end of day 17 to end of day 18, end of day 18 to end of day 19, end of day 19 to end of day 20, end of day 20 to end of day 21, end of day 21 to end of day 22, end of day 22 to end of day 23, end of day 23 to end of day 24, end of day 24 to end of day 25, end of day 25 to end of day 26, end of day 26 to end of day 27, end of day 27 to end of day 28, or any cumulative time period of any of the aforementioned time periods, after administration of the drug delivery system to a subject. In some embodiments, the average drug release is measured from after 24 hr to up to 26 days after administration of the drug delivery device to a subject. As used herein, the term “average” refers to the mean average (total values of released specified drug divided by number of days).

In some embodiments, this disclosure comprises a method of contraception. This method comprises a step of retainably positioning the drug delivery system, e.g., the vaginal ring, within a subject's vaginal tract; retaining the system within the subject's vaginal tract for a period of time; and removing the system from the subject after the period of time (or total period of time). In some embodiments, the period of time (or total period of time) is about 21 days. In some embodiments, the period of time (or total period of time) is about 24 days. In some embodiments, the period of time (or total period of time) is about 26 days. In some embodiments, the period of time (or total period of time) is about 3 months. In yet some embodiments, the method can include intermittent removal and reinsertion of the drug delivery system at prescribed intervals for prescribed periods of time.

In some embodiments, the period of time (or total period of time) is about 26 days and removal for 2 days within a 28-day menstrual cycle.

In some embodiments, the drug delivery system releases on an average about from about 0.001 to about 0.013 mg of ethinyl estradiol per day for about 21 days after administration to the subject's vaginal tract. In some embodiments, the drug delivery system releases on an average about from about 0.001 to about 0.013 mg of ethinyl estradiol per day for about 24 days after administration to the subject's vaginal tract. In some embodiments, the drug delivery system releases on an average about from about 0.001 to about 0.013 mg of ethinyl estradiol per day for about 26 days after administration to the subject's vaginal tract.

In some embodiments, the drug delivery system releases on an average about 0.001, 0.002, 0.003, 0.004, 0.005, 0.006, 0.007, 0.008, 0.009, 0.010, 0.011, 0.012, and 0.013 mg, or any value between the aforementioned values, of ethinyl estradiol per day, or any value between the aforementioned values, for about 21 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.011, 0.012, and 0.013 mg, or any value between the aforementioned values, of ethinyl estradiol per day for about 24 days after administration to the subject's vaginal tract. In some embodiments, the drug delivery system releases on an average about 0.001, 0.002, 0.003, 0.004, 0.005, 0.006, 0.007, 0.008, 0.009, 0.010, 0.011, 0.012, and 0.013 mg, or any value between the aforementioned values, of ethinyl estradiol per day, or any value between the aforementioned values, for about 26 days after administration to the subject's vaginal tract.

In some embodiments, the drug delivery system releases on an average about 0.013 mg per day of ethinyl estradiol to the subject for about 21 days after administration. In some embodiments, the drug delivery system releases on an average about 0.013 mg per day of ethinyl estradiol to the subject for about 24 days after administration. In some embodiments, the drug delivery system releases on an average about 0.013 mg per day of ethinyl estradiol to the subject for about 26 days after administration.

In a preferred embodiment, the system releases on an average about 0.010 mg of ethinyl estradiol per day for about 21 days after administration to the subject's vaginal tract, after the first day of administration. In another preferred embodiment, the system releases on an average about 0.010 mg of ethinyl estradiol per day for about 24 days after administration to the subject's vaginal tract. In another preferred embodiment, the system releases on an average about 0.010 mg of ethinyl estradiol per day for about 26 days after administration to the subject's vaginal tract.

In some embodiments, the drug delivery system releases on an average about 0.010 mg per day of ethinyl estradiol to the subject for about 26 days after administration.

In some embodiments, the drug delivery system releases on an average about from about 0.1 mg to about 0.2 mg of etonogestrel per day for about 21 days. In some embodiments, the drug delivery system releases on an average about from about 0.1 mg to about 0.2 mg of etonogestrel per day for about 24 days. In some embodiments, the drug delivery system releases on an average about from about 0.1 mg to about 0.2 mg of etonogestrel per day for about 26 days. In some embodiments, the drug delivery system releases on an average about from about 0.1 mg to about 0.2 mg of etonogestrel per day for about 3 months.

In some embodiments, the system releases on an average from about 0.1 mg to about 0.15 mg of etonogestrel per day for about 21 days after administration to the subject's vaginal tract. In some embodiments, the system releases on an average from about 0.1 mg to about 0.15 mg of etonogestrel per day for about 24 days after administration to the subject's vaginal tract. In some embodiments, the system releases on an average from about 0.1 mg to about 0.15 mg of etonogestrel per day for about 26 days after administration to the subject's vaginal tract.

In some embodiments, the system releases on an average from about 0.1 mg to about 0.12 mg of etonogestrel per day for about 21 days after administration to the subject's vaginal tract. In some embodiments, the system releases on an average from about 0.1 mg to about 0.12 mg of etonogestrel per day for about 24 days after administration to the subject's vaginal tract. In some embodiments, the system releases on an average from about 0.1 mg to about 0.12 mg of etonogestrel per day for about 26 days after administration to the subject's vaginal tract.

In some embodiments, the drug delivery system releases on an average about 0.10, 0.11, 0.12, 0.13, 0.14, 0.15, 0.16, 0.17, 0.18, 0.19 or 0.20 mg, or any value between the aforementioned values, of etonogestrel per day, or any value between the aforementioned values, for about 21 days after administration to the subject's vaginal tract. In some embodiments, the drug delivery system releases on an average about 0.10, 0.11, 0.12, 0.13, 0.14, 0.15, 0.16, 0.17, 0.18, 0.19 or 0.20 mg of etonogestrel per day, or any value between the aforementioned values, for about 24 days after administration to the subject's vaginal tract. In some embodiments, the drug delivery system releases on a mean average about 0.10, 0.11, 0.12, 0.13, 0.14, 0.15, 0.16, 0.17, 0.18, 0.19 or 0.20 mg, or any value between the aforementioned values, of etonogestrel per day, or any value between the aforementioned values, for about 26 days after administration to the subject's vaginal tract.

In some embodiments, the system releases on an average about 0.12 mg of etonogestrel per day for about 21 days after administration to the subject's vaginal tract. In another preferred embodiment, the system releases on an about 0.12 mg of etonogestrel per day for about 24 days after administration to the subject's vaginal tract. In another preferred embodiment, the system releases on an average about 0.12 mg of etonogestrel per day for about 26 days after administration to the subject's vaginal tract. In another preferred embodiment, the system releases on an average about 0.12 mg of etonogestrel per day for about 3 months after administration to the subject's vaginal tract.

In some embodiments, the drug delivery system releases on an average from about 0.001 to about 0.013 mg per day of ethinyl estradiol and on an average from about 0.10 to about 0.12 mg of etonogestrel per day for about 21 days after administration to the subject's vaginal tract. In some embodiments, the drug delivery system releases on an average from about 0.001 to about 0.013 mg per day of ethinyl estradiol and on an average from about 0.10 to about 0.12 mg of etonogestrel per day for about 24 days after administration to the subject's vaginal tract. In some embodiments, the drug delivery system releases on an average from about 0.001 to about 0.013 mg per day of ethinyl estradiol and on an average from about 0.10 to about 0.12 mg of etonogestrel per day for about 26 days after administration to the subject's vaginal tract.

In some embodiments, the drug delivery system releases on an average about 0.010 mg per day of ethinyl estradiol and on an average about 0.12 mg of etonogestrel per day for about 21 days after administration to the subject's vaginal tract. In some embodiments, the drug delivery system releases on an average about 0.010 mg per day of ethinyl estradiol and on an average about 0.12 mg of etonogestrel per day for about 24 days after administration to the subject's vaginal tract. In some embodiments, the drug delivery system releases on an average about 0.010 mg per day of ethinyl estradiol and on an average about 0.12 mg of etonogestrel per day for about 26 days after administration to the subject's vaginal tract.

In some embodiments, the system delivers about 0.010 mg per day of ethinyl estradiol and about 0.12 mg of etonogestrel per day for about 26 days after administration to the subject's vaginal tract.

In some embodiments, the drug delivery system comprises from about 0.1 mg to about 7.5 mg ethinyl estradiol. In some embodiments, the drug delivery system comprises from about 0.1 mg to about 5 mg ethinyl estradiol. In some embodiments, the drug delivery system comprises from about 0.1 mg to about 2.5 mg ethinyl estradiol. In some embodiments, the drug delivery system comprises from about 0.1 mg to about 2.0 mg ethinyl estradiol. In some embodiments, the drug delivery system comprises from about 0.1 mg to about 1.5 mg ethinyl estradiol.

In some embodiments, the drug delivery system comprises about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 mg, or any value between the aforementioned values, of ethinyl estradiol.

In some embodiments, the drug delivery system comprises about 1.0 to 1.4 mg of ethinyl estradiol. In some embodiments, the drug delivery system comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 mg, or any value between the aforementioned values, of ethinyl estradiol. In some embodiments, the drug delivery system comprises about 1.27 mg of ethinyl estradiol.

In some embodiments, the drug delivery system comprises from 0.4 to 0.6 wt. % of etonogestrel relative to the total weight of the drug delivery system. In some embodiments, the drug delivery system comprises about 0.5 wt. % of etonogestrel.

In some embodiments, the drug delivery system comprises from 0.05 to 0.1 wt. % of ethinyl estradiol relative to the total weight of the drug delivery system. In some embodiments, the drug delivery system comprises about 0.07 wt. % of ethinyl estradiol.

In some embodiments, the drug delivery system comprises about 0.07 wt. % of ethinyl estradiol and about 0.5 wt. % of etonogestrel.

In some embodiments, the drug delivery system comprises from about 1 mg etonogestrel to about 75 mg etonogestrel. In some embodiments, the drug delivery system comprises about 5 mg etonogestrel to about 12 mg etonogestrel. In some embodiments, the drug delivery system comprises about 5 mg etonogestrel to about 11 mg etonogestrel. In some embodiments, the drug delivery system comprises about 5 mg etonogestrel to about 10 mg etonogestrel. In some embodiments, the drug delivery system comprises about 5 mg etonogestrel to about 9 mg etonogestrel. In some embodiments, the drug delivery system comprises about 5 mg etonogestrel to about 8 mg etonogestrel. In some embodiments, the drug delivery system comprises about 5 mg etonogestrel to about 7 mg etonogestrel.

In some embodiments, the drug delivery system comprises about 5, 10, 15, 20, 25, 30, 35, 40, 45, 50, 55, 60, 65, 70, or 75 mg, or any value between the aforementioned values, of etonogestrel. In some embodiments, the drug delivery system comprises about 5, 6, 7, 8, 9, 10, 11, 12 mg, or any value between the aforementioned values, of etonogestrel. In some embodiments, the drug delivery system comprises about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0 mg, or any value between the aforementioned values, of etonogestrel.

In some embodiments, the drug delivery system comprises about 8.5 mg of etonogestrel.

In some embodiments, the drug delivery system comprises about 0.1 mg to about 2.5 mg of ethinyl estradiol and about 5 mg to about 12 mg of etonogestrel. In some embodiments, the drug delivery system comprises about 0.5 mg to about 2.0 mg of ethinyl estradiol and about 7 mg to about 11 mg of etonogestrel. In some embodiments, the drug delivery system comprises about 0.5 mg to about 1.5 mg of ethinyl estradiol and about 8 mg to about 11 mg of etonogestrel. In some embodiments, the drug delivery system comprises about 0.5 mg to about 1.5 mg of ethinyl estradiol and about 8 mg to about 10 mg of etonogestrel.

In some embodiments, the drug delivery system comprises about 8.50 mg of etonogestrel and about 1.27 mg of ethinyl estradiol.

In a preferred embodiment, the drug delivery system comprises about 8.5 mg of etonogestrel and about 1.27 mg of ethinyl estradiol. In some embodiments, the drug delivery system delivers an in-vitro release of an average of about 0.12 mg per day of Etonogestrel and 0.010 mg per day of Ethinyl Estradiol for a period of about 26 days. In some embodiments, the delivery release average is calculated after the initial 24 hr burst period following administration of the drug delivery device to the subject.

In some embodiments, the core and the membrane are each independently made of a thermoplastic polymer or an elastomer.

In some embodiments, the core is made of a material selected from the group consisting of low-density polyethylene, ethylene-vinyl acetate copolymers, thermoplastic polyurethane, styrene-butadiene-styrene copolymers, and a combination thereof.

In some embodiments, the membrane is made of a material selected from the group consisting of olefins, vinyl polymers, rubber polymers, silicon polymers, microporous polymers, diffusion polymers, polyethylene, ethylene-vinyl acetate copolymers, thermoplastic polyurethanes, polyether-ester polymers, cellulose, and a combination thereof.

In some embodiments, the core is made of an ethylene-vinyl acetate copolymer having a vinyl acetate (VA) relative molar content from 25 to 35%. In some embodiments, the core is made of an ethylene-vinyl acetate copolymer having a vinyl acetate relative molar content from 27 to 29%. In some embodiments, the core is made of an ethylene-vinyl acetate copolymer having a vinyl acetate relative molar content particularly of 28%. “Relative molar content” or “relative content” when referring to polymer identity, for the copolymers described herein means the relative moles of each of the comonomers added to the polymerization reaction to produce the copolymer. The copolymers described herein are referred to by their relative molar content percentage.

In some embodiments, the membrane is made of an ethylene-vinyl acetate copolymer having a vinyl acetate relative molar content from 8 to 10%. In some embodiments, the membrane is made of an ethylene-vinyl acetate copolymer having a vinyl acetate relative molar content of 9%.

In some embodiments, the core of the drug delivery system comprises of an ethylene-vinyl acetate copolymer with 28% vinyl acetate relative molar content from about 81 wt. % to about 92 wt. % relative to the total weight of the drug delivery system. In some embodiments, the core of the drug delivery system comprises of an ethylene-vinyl acetate copolymer with 28% vinyl acetate content from about 85 wt. % to about 87 wt. % relative to the total weight of the drug delivery system.

In some embodiments, the membrane of the drug delivery system comprises of an ethylene-vinyl acetate copolymer with 9% vinyl acetate relative molar content from about 11.7 wt. % to about 14.3 wt. % relative to the total weight of the drug delivery system. In some embodiments, the membrane of the drug delivery system comprises of an ethylene-vinyl acetate copolymer with 9% vinyl acetate relative molar content from about 12.5 wt. % to about 13.5 wt. % relative to the total weight of the drug delivery system.

In some embodiments, the membrane further comprises a plasticizer to modulate the release rate of the etonogestrel and/or ethinyl estradiol. The plasticizer can be selected from fatty acids (and esters thereof), pyrrolidones, propylene glycol derivatives, glycerides, and a non-ionic surfactant (Cho et al., Iran J Pharm Res. 2012 Winter; 11(1): 3-12).

In some embodiments, the ring has an outer diameter of from about 50 millimeters to about 60 millimeters, or an outer diameter of from about 52 millimeters to about 56 millimeters. In some embodiments, the ring has an outer diameter of from about 52 millimeters to about 55 millimeters. In some embodiments, the ring has an outer diameter of from about 52 millimeters to about 54 millimeters. In some embodiments, the ring has an outer diameter of about 52 millimeters. In some embodiments, the ring has an outer diameter of about 53 millimeters. In some embodiments, the ring has an outer diameter of about 53.5 millimeters. In some embodiments, the ring has an outer diameter of about 54 millimeters. In some embodiments, the ring has an outer diameter size between any of the aforementioned diameter sizes.

In some embodiments, the ring has an inner diameter of from about 44 millimeters to about 47 millimeters. In some embodiments, the ring has an inner diameter of from about 45 millimeters to about 46 millimeters. In some embodiments, the ring has an inner diameter of about 44 millimeters. In some embodiments, the ring has an inner diameter of about 45 millimeters. In some embodiments, the ring has an inner diameter of about 45.8 millimeters. In some embodiments, the ring has an inner diameter of about 46 millimeters. In some embodiments, the ring has an inner diameter size between any of the aforementioned diameter sizes.

In some embodiments, the ring has a cross-sectional diameter of from about 2.5 millimeters to about 5 millimeters. In some embodiments, the ring has a cross-sectional diameter of from about 3.0 millimeters to about 4 millimeters. In some embodiments, the ring has a cross-sectional diameter of about 3.0 millimeters. In some embodiments, the ring has a cross-sectional diameter of about 3.9 millimeters. In some embodiments, the ring has a cross-sectional diameter of about 4 millimeters. In some embodiments, the ring has a cross-sectional diameter size between any of the aforementioned cross-sectional diameter sizes.

In some embodiments, the ring has total weight ranging from about 1.4 g to about 2.5 g. In some embodiments, the ring has a total weight ranging from about 1.6 g to about 1.8 g. In some embodiments, the ring has a total weight of about 1.71 g.

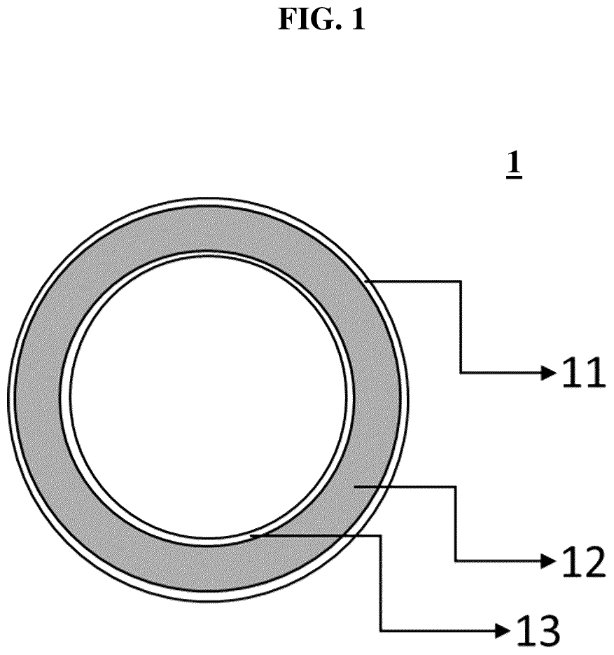

The drug delivery system, e.g., as shown in FIG. 1, can be in the form of a vaginal ring. The vaginal rings of this invention have the advantage that the active agents are released therefrom for a longer period of time (or total time period) regularly and uniformly within the limits of the dosage required for the desired biological effect, e.g. for the inhibition of ovulation. Moreover, these vaginal rings have the advantage that the amount of active agent which must be incorporated into a single such contraceptive can be much lower than in the case of the conventional vaginal rings.

In some embodiments, the vaginal ring can be an annular ring that comprises a core (12) and an outer skin (also called membrane) (11 and 13) as shown by the exemplary drug delivery system (1) in FIG. 1. The active agents described in the invention are included in the core (12) of the ring and are in a form whereby they can be released from the core (12) of the ring to the subject's vaginal tract upon administration of the ring to the subject's vaginal tract. For instance, when the ring as shown in FIG. 1 is administered to the subject, the ring releases or delivers the active ingredients to the subject's vaginal tract. The skin (11 and 13) can be made such that it controls the release rate of the active agents included within the core (12) of the ring. In some embodiments, the active agents included in the vaginal ring are released from the core (12) of the drug delivery system (1) such that they are absorbed by the vaginal mucosa of the subject.

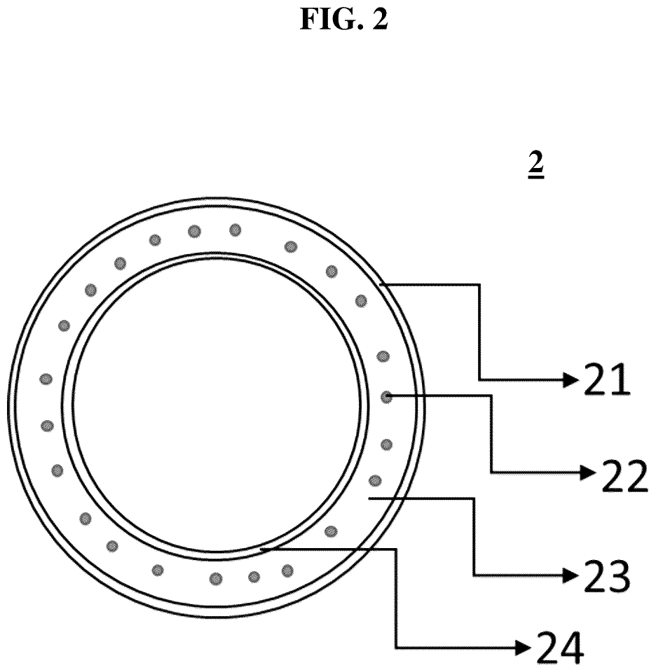

In some embodiments, the drug delivery system is in the form of a ring, as shown by the exemplary drug delivery system (2) in FIG. 2, where the core (23) comprises the active agents in the form of granules, beads, spheres, crystals or the like (22). The granules of the active agent are such that they are capable of delivering the active agent to a subject upon administration of the ring to the subject's vaginal tract. The core (23) of the ring may be optionally covered by a membrane (21 and 24). The membrane controls the release rate of the active agents included within the core (23) of the ring in exemplary drug delivery system (2).

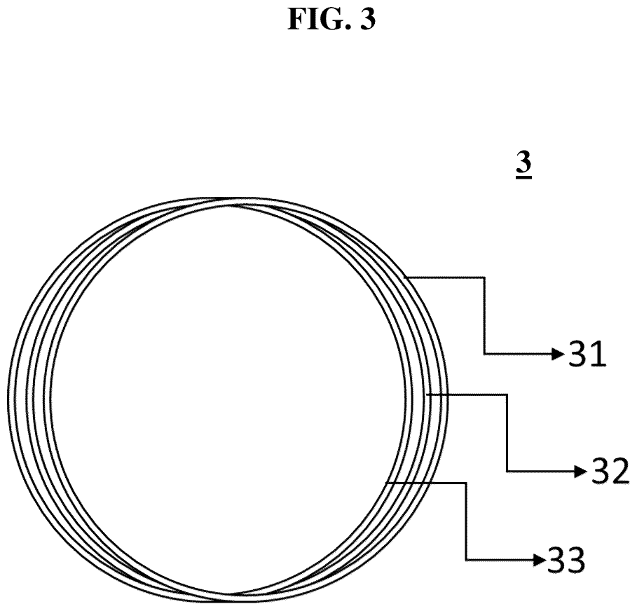

In some embodiments, the drug delivery system is in the form of multiple annular rings that may be attached to one another, concatenated, or connected to one another, as shown by the exemplary drug delivery system (3) in FIG. 3. One or more rings (31, 32, and 33) may include a separate active agent dispersed within the one or more rings (31, 32 and 33, respectively). One or more rings (31, 32, and 33) may optionally include a membrane over the surface of the central core portion of the one or more rings (31, 32, and 33) where the membrane controls the rate of release of the active agent included within the one or more rings (31, 32, and 33).

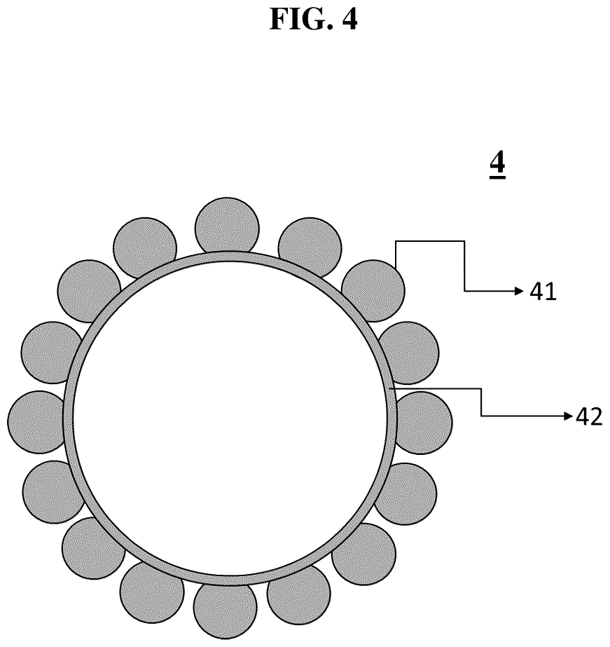

In some embodiments, the vaginal ring comprises a retaining annular ring (42) in the center and a plurality of pockets or compartments (41) attached to the outer portion the retaining annular ring (42), as shown by the exemplary drug delivery system (4) in FIG. 4. Each pocket or compartment (41) as well as the retaining annular ring (42) may include an active ingredient and is such that the active ingredient may be delivered to the subject upon administration of the vaginal ring to the subject's vaginal tract.

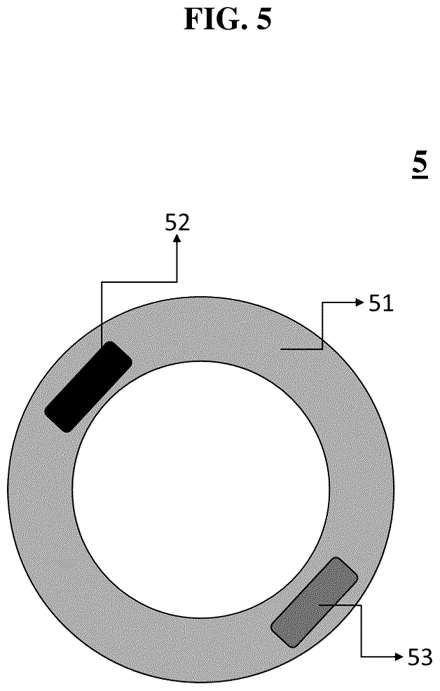

In some embodiments, the vaginal ring comprises a retaining annular ring (51) and a plurality of pockets or compartments (52, 53) within the retaining annular ring (51), as shown by the exemplary drug delivery system (5) in FIG. 5. Each pocket or compartment (52, 53) may include an active ingredient and is such that the active ingredient may be delivered to the subject upon administration of the vaginal ring to the subject's vaginal tract.

In some embodiments, the drug delivery systems of the invention may be in the shape or form of the vaginal rings disclosed in U.S. Pat. Nos. 3,995,633; 3,995,634; 4,292,965; 4,596,576; and 5,989,581, all of which are hereby incorporated by reference in their entirety.

Manufacturing Process:

The drug delivery systems of this disclosure provide reliable and predictable release of the steroid compounds and active agents of the invention. In some embodiments, the drug delivery system comprises a thermoplastic polymer core or an elastomer core, optionally, containing at least a steroidal progestogenic compound and a steroidal estrogenic compound.

The polymer core allows direct release of both the progestogenic compound and the estrogenic compound in physiologically required amounts. In some embodiments, the progestogenic compound is dissolved in the core polymer at a relatively low degree of supersaturation, preferably between about 1 to about 6 times of the amount by weight necessary for obtaining the saturation concentration of said progestogenic steroid in said core polymer at 25° C. In some embodiments, the estrogenic compound is dissolved in the core polymer at a concentration that is lower than that of the said progestogenic compound. The drug delivery system, in some embodiments, has a thermoplastic skin or membrane (outer layer) that is permeable to the progestogenic and estrogenic compounds.

The thermoplastic polymer that can be used in practicing the invention may in principle be any thermoplastic polymer or elastomer material suitable for pharmaceutical use, such as ethylene-vinyl acetate copolymers, low-density polyethylene, styrene-butadiene-styrene copolymers, thermoplastic polyurethanes or a combination thereof. The ethylene-vinyl acetate copolymer (EVA copolymer) is highly preferred due to its excellent mechanical and physical properties (e.g., solubility of the steroids in the material). The EVA copolymer material may preferably be used for both the core as well as the membrane/skin and can be any commercially available ethylene-vinyl acetate copolymer, such as the products available under the trade names: Ativa®, VitalDose®, Elvax®, Evatane®, Lupolen®, Movriton™, Ultrathene® and Vestypar®. The membrane/skin of the vaginal ring can, e.g., also be made of olefins, vinyl polymers, rubber polymers, silicon polymers, microporous polymers, diffusion polymers, polyethylene, ethylene-vinyl acetate copolymers, polyether-ester polymers, cellulose, or combinations thereof.

The membrane or skin, as disclosed in the invention, has excellent solubility and steroid diffusion properties, allowing for release of the steroidal estrogenic compound, the steroidal progestogenic compound, or a combination thereof. In a preferred embodiment, the skin/membrane allows for the combined release of the steroidal estrogenic compound and the steroidal progestogenic compound in the proper ratio at moderate concentrations of the steroids in the vaginal ring during a period of time (or total time period).

The drug delivery system according to the invention can have any size, shape, and form as required by, e.g., its function, its use, and anatomy of a subject who is a recipient of the drug delivery system. In a preferred embodiment, the drug delivery system has a size, shape and form that are adapted for easy placement in, retention in and easy removal from a subject's vaginal tract.

In another preferred embodiment, the drug delivery system according to the invention can be manufactured in any size as required. In some embodiments, the drug delivery system is a ring-shape. In some embodiments, the ring has an outer diameter of between about 50 and about 60 mm and more preferably between about 52 and about 56 mm; the cross sectional diameter of the vaginal ring is preferably between about 2.5 and about 7.5 mm.

In some embodiments, the ring-shaped drug delivery systems have a circular cross section. In some embodiments, the cross section of the ring-shaped drug delivery system comprises a shape selected from a square, rectangle, oval, figure-eight, triangle, or a combination thereof. In some embodiments, the dimensions of the drug delivery system are selected so that it maintains contact points with the vaginal mucosa or the fluid present in the vaginal tract of the subject upon administration the subject's vaginal tract.

The size of the drug delivery system is dependent on the species for utilization of the ring, e.g., for smaller mammals, such as dogs, the ring size will be smaller than in the case of larger mammals such as horses and cows. In vaginal rings for use in women, the outer diameter is between about 50 to about 60 mm, and in the case of Rhesus monkeys, for example, between about 20 to 30 mm. The diameter of such ring cross sections is generally between about 2.5 to about 7.5 mm.

The drug delivery system according to the invention can be manufactured in any suitable manner know in the art, e.g., like the ones disclosed in e.g. U.S. Pat. Nos. 3,995,633; 3,99,634; 4,292,965; 4,596,576; and 5,989,581, all of which are hereby incorporated by reference in their entirety. A preferred method of manufacture of the vaginal rings comprises co-extrusion of a drug-loaded core and the non-medicated outer layer. The fibers thus obtained are cut into pieces of the required length and each piece is assembled to a ring shaped device in any suitable manner. The rings are then packed for example in a suitable sachet, optionally, after being sterilized or disinfected.

In some embodiments the drug delivery systems of this disclosure can be made using an injection molding and lamination method. Etonogestrel and Ethinyl Estradiol compounds are mixed with 28% VA-content EVA copolymer, then extruded. The extruded strands were then pelletized. The pellets were injection molded into discs. The discs were compressed and then laminated using thermoformed sheath discs comprising 9% VA-content EVA copolymer.

In some embodiments, the drug delivery systems of this disclosure can be made using a coaxial extrusion manufacturing process which comprises mixing Etonogestrel and Ethinyl Estradiol compounds with 28% VA-content EVA copolymer as an outer layer, then co-extruding the mixture with 9% VA-content EVA copolymer to form a co-axial fiber. Finally, the two ends of each fiber piece are joined together (using, e.g., solvent or heat-welding).

Methods of Contraception

In some embodiments, this disclosure provides for a method of contraception. This method comprises a step of retainably positioning the drug delivery system, e.g., the vaginal ring within a subject's vaginal tract; retaining the system within the subject's vaginal tract for a period of time; and removing the system from the subject after the period of time (or total period of time). In some embodiments, the period of time (or total period of time) is about 21 days. In some embodiments, the period of time (or total period of time) is about 24 days. In some embodiments, the period of time (or total period of time) is about 26 days. In some embodiments, the period of time (or total period of time) is about 3 months. In yet some embodiments, the method can include intermittent removal and reinsertion of the drug delivery system at prescribed intervals for prescribed periods of time.

Vaginal rings of the invention are, in one instance, meant to be introduced into the vagina tract of a subject in a simple manner without medical assistance. The vaginal ring fits between the rear wall of the vagina and the upper edge of the pubic bone. The vaginal ring of the invention is primarily useful for the inhibition of fertility, which is contraceptive purposes, but in some instances may also be used to treat and medicate other conditions. The vaginal rings are designed so that they can be retained in the vagina for a period about 21 days to about one year. In some embodiments, the ring is inserted in the vagina for 3 weeks, removed for one week and reinserted on a schedule of three weeks in, one week out, for a period of time between 1 month to 1 year, 2 months to 6 months, or, more preferably for 3 months.

The vaginal rings contain medication or active agents, for example, an effective amount of contraceptive steroids, such as steroidal estrogenic compounds or steroidal progestogenic compounds, which diffuse through the vaginal rings and are absorbed by the surrounding body fluid through the vaginal mucosa or by via the vaginal mucosa. The vaginal ring exerts its medicative or contraception effect as long as the vaginal ring is retained within the body or the supply of the medication in the vaginal ring or the subject's body is sufficient for its indicated purpose. The concept of using vaginal rings as a means of administering effective contraceptive steroids by absorption through the vaginal mucosa for contraceptive purposes was tested by Mishell and associates in 1970, Mishell, D. R. Jr., et al. Am. J. Obstet. Gynecol. 107:100, 1970, which is hereby incorporated by reference in its entirety.

In some embodiments, this invention is related to a method of making the drug delivery system. This method comprises a step of producing a core comprising the combination of a steroidal estrogenic compound and a steroidal progestogenic compound and a subsequent step of covering a portion or all of the (the entire) core with a membrane or a skin to form the drug delivery system. In some embodiments, the core of the drug delivery system is coextruded with the membrane or the skin to form the drug delivery system.

The vaginal ring according to the invention can be manufactured in any suitable manner, e.g., as disclosed in U.S. Pat. Nos. 5,989,581; 4,012,496; and 4,292,965. A preferred method of manufacture comprises co-extrusion of the drug-loaded core and the non-medicated outer layer. The fibers thus obtained are cut into pieces of the required length and each piece is assembled to a ring shaped device in any suitable manner. The rings are then packed for example in a suitable sachet, optionally after being sterilized or disinfected.

The vaginal ring according to the invention is primarily designed for contraceptive use, but as said above may also be used under certain conditions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variations of certain described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention comprises all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the invention. Other modifications that may be employed are within the scope of the invention. Thus, by way of example, but not of limitation, alternative configurations of the invention may be utilized in accordance with the teachings herein. Accordingly, the invention is not limited to that precisely as shown and described.

### EXAMPLES

### Example 1: Formulation of Representative Drug Delivery System of the Invention

The formulation batch was manufactured using 8.50 mg Etonogestrel and 1.27 mg Ethinyl Estradiol per unit. The compounds were mixed with the 28% VA-content EVA copolymer and co-extruded by the methods described herein. Next, the extruded strands were pelletized. The pellets were injection molded into discs to form core rings. The core rings were then laminated using thermoformed sheath discs of 9% VA-content EVA copolymer. The formulation details are presented in the Table 1, as shown below:

TABLE 1
Composition of a representative embodiment
vaginal ring of this disclosure.

| Ingredient | mg/unit | % w/w |
|---|---|---|
| Etonogestrel | 8.50 | 0.50 |
| Ethinyl Estradiol | 1.27 | 0.07 |
| 28% VA-content EVA copolymer | 1474.33 | 86.38 |
| Core Unit Weight | 1484.10 | 86.96 |
| 9% VA-content EVA | 222.60 | 13.04 |
| Copolymer Sheath | | |
| Total Ring Weight | 1706.70 | 100.00 |

### Example 2: In-vitro Drug Release Measurements from Drug Delivery System of Example 1

The in-vitro release rate was determined for a vaginal ring of Example 1 as a representative embodiments of the invention and compared to that of the NuvaRing® as a reference product. The vaginal rings were placed submerged in jars containing 100 mL of elution media. The jars were then placed on a shaker table at 120 rpm and at a temperature of 37° C. The samples were collected on Day 1, 3, 7, 10, 14, 17, 21, 24, and 26 (or closest day possible). To collect samples, the ring was completely removed from the jar, dabbed dry, and placed in a new jar containing fresh media at target temperature, which was placed back on the shaker until the next pull day. The detection of the Etonogestrel and Ethinyl Estradiol in the in-vitro release medium was performed with HPLC. Elution Media was prepared by preparing 1 L of a 22 mM sodium acetate buffer solution by dissolving approximately 3 g of sodium acetate in 1 L of purified water. Adjust pH to 4.5 using glacial acetic acid (GAA). Add 10 g of sodium dodecyl sulfate and mix well.

The results for in-vitro release are shown in Tables 2 and 3. The average release for NuvaRing® is calculated based on the release rate of day 3 to 24. An arithmetic mean of the individual release rates for NuvaRing® is obtained between and including the days 3 and 24. The average release for the vaginal rings of Example 1 is also calculated based on release rate of day 3 to 24. An arithmetic mean of the individual release rates for the vaginal rings of Example 1 is obtained between and including the days 3 and 24.

TABLE 2
Release of Etonogestrel from vaginal rings produced from Example 1 in
comparison to NuvaRing ®

| | | | | | | | | | | Average |
|---|---|---|---|---|---|---|---|---|---|---|
| | Time Point | | | | | | | | | Day |
| Replicate | 1 | 3 | 7 | 10 | 14 | 17 | 21 | 24 | 26 | 3-24 |

Release of Etonogestrel from Example 1 ring (micrograms (μg))

| | | | | | | | | | | |
|---|---|---|---|---|---|---|---|---|---|---|
| 1 | 274.01 | 159.39 | 138.88 | 130.02 | 120.28 | 110.02 | 102.66 | 95.63 | 92.44 | 122.41 |
| 2 | 279.77 | 177.45 | 154.06 | 141.66 | 130.08 | 117.75 | 109.26 | 100.57 | 97.25 | 132.98 |
| 3 | 281.21 | 175.32 | 153.20 | 130.69 | 120.35 | 110.49 | 103.80 | 96.49 | 92.80 | 127.19 |
| 4 | 249.10 | 160.77 | 141.56 | 127.34 | 118.08 | 108.10 | 102.33 | 95.02 | 91.00 | 121.89 |
| 5 | 266.31 | 169.93 | 146.24 | 136.07 | 125.95 | 114.43 | 107.24 | 98.89 | 95.80 | 128.39 |
| 6 | 260.06 | 171.80 | 150.31 | 135.89 | 124.83 | 113.85 | 106.18 | 98.57 | 95.17 | 128.78 |
| 7 | 267.23 | 178.81 | 148.89 | 118.44 | 110.19 | 101.43 | 96.39 | 92.24 | 89.18 | 120.91 |
| 8 | 297.52 | 174.42 | 154.73 | 126.63 | 117.24 | 108.22 | 102.22 | 96.89 | 92.79 | 125.76 |
| 9 | 262.79 | 171.40 | 139.91 | 125.42 | 115.93 | 106.77 | 101.30 | 95.82 | 91.49 | 122.36 |
| 10 | 292.99 | 176.20 | 144.36 | 128.26 | 117.67 | 106.18 | 101.98 | 96.72 | 92.80 | 124.48 |
| 11 | 270.92 | 163.88 | 151.37 | 122.32 | 113.77 | 105.02 | 98.43 | 94.73 | 93.00 | 121.36 |
| 12 | 269.97 | 161.13 | 149.55 | 125.28 | 116.18 | 107.22 | 100.70 | 94.68 | 92.75 | 122.11 |

Release of Etonogestrel from NuvaRing ® (micrograms (μg))

| | | | | | | | | | | |
|---|---|---|---|---|---|---|---|---|---|---|
| 1 | 233.17 | 171.05 | 155.64 | 139.32 | 128.20 | 119.97 | 114.91 | 109.06 | 105.84 | 134.02 |
| 2 | 249.74 | 185.14 | 162.15 | 150.03 | 137.67 | 129.93 | 121.42 | 114.60 | 110.79 | 142.99 |
| 3 | 256.27 | 192.21 | 166.07 | 153.29 | 142.38 | 131.88 | 123.07 | 116.35 | 112.66 | 146.46 |
| 4 | 244.00 | 189.59 | 165.49 | 151.84 | 141.90 | 125.76 | 120.39 | 114.85 | 110.86 | 144.26 |
| 5 | 249.24 | 175.37 | 156.96 | 143.35 | 133.09 | 123.52 | 118.35 | 112.18 | 108.15 | 137.55 |
| 6 | 262.23 | 185.70 | 164.56 | 149.98 | 145.43 | 130.84 | 121.98 | 115.25 | 111.30 | 144.82 |

TABLE 3
Release of Ethinyl Estradiol from vaginal rings produced from Example 1 in comparison
to NuvaRing ®

| | | | | | | | | | | Average |
|---|---|---|---|---|---|---|---|---|---|---|
| | Time Point (Day) | | | | | | | | | Day |
| Replicate | 1 | 3 | 7 | 10 | 14 | 17 | 21 | 24 | 26 | 3-24 |

Release of Ethinyl Estradiol from Example 1 ring (micrograms (μg))

| | | | | | | | | | | |
|---|---|---|---|---|---|---|---|---|---|---|
| 1 | 24.89 | 13.01 | 10.82 | 10.64 | 9.89 | 9.48 | 8.96 | 8.72 | 8.66 | 10.22 |
| 2 | 25.14 | 14.97 | 12.34 | 11.33 | 11.12 | 10.39 | 9.87 | 9.53 | 9.40 | 11.36 |
| 3 | 25.89 | 14.43 | 11.78 | 10.88 | 10.17 | 9.17 | 9.25 | 8.96 | 8.81 | 10.66 |
| 4 | 21.96 | 13.11 | 10.63 | 10.58 | 10.02 | 9.47 | 9.16 | 8.77 | 8.66 | 10.25 |
| 5 | 23.89 | 14.04 | 11.40 | 11.32 | 10.65 | 9.90 | 9.45 | 8.94 | 9.07 | 10.81 |
| 6 | 23.32 | 14.29 | 11.28 | 11.29 | 10.49 | 10.03 | 9.52 | 9.18 | 9.05 | 10.87 |
| 7 | 23.71 | 14.25 | 12.18 | 9.92 | 9.54 | 8.59 | 8.37 | 8.05 | 8.55 | 10.13 |
| 8 | 26.88 | 13.76 | 12.40 | 10.34 | 9.92 | 9.05 | 9.05 | 8.28 | 8.84 | 10.40 |
| 9 | 23.08 | 13.33 | 11.18 | 10.36 | 9.86 | 9.23 | 9.05 | 8.17 | 8.79 | 10.17 |
| 10 | 26.63 | 13.43 | 11.20 | 10.77 | 10.14 | 9.08 | 9.17 | 8.61 | 8.94 | 10.34 |
| 11 | 23.62 | 12.99 | 12.15 | 10.03 | 9.67 | 8.45 | 8.81 | 7.70 | 8.71 | 9.97 |
| 12 | 25.45 | 13.01 | 12.60 | 10.89 | 10.31 | 9.49 | 9.45 | 8.30 | 9.32 | 10.58 |

Release of Ethinyl Estradiol from NuvaRing ® (micrograms (μg)

| | | | | | | | | | | |
|---|---|---|---|---|---|---|---|---|---|---|
| 1 | 29.10 | 19.94 | 18.26 | 15.48 | 15.77 | 16.44 | 15.16 | 14.86 | 14.67 | 16.56 |
| 2 | 30.83 | 21.55 | 18.55 | 18.43 | 16.08 | 17.60 | 16.33 | 15.38 | 15.65 | 17.70 |
| 3 | 31.54 | 22.39 | 19.24 | 17.64 | 17.71 | 17.70 | 16.40 | 16.12 | 16.38 | 18.17 |
| 4 | 29.49 | 21.02 | 20.11 | 18.30 | 17.55 | 17.03 | 16.01 | 15.75 | 15.44 | 17.97 |
| 5 | 31.10 | 20.43 | 18.03 | 18.05 | 16.59 | 16.84 | 15.58 | 15.41 | 14.90 | 17.28 |
| 6 | 32.03 | 20.05 | 19.93 | 17.46 | 18.47 | 17.55 | 16.08 | 16.06 | 15.50 | 17.94 |

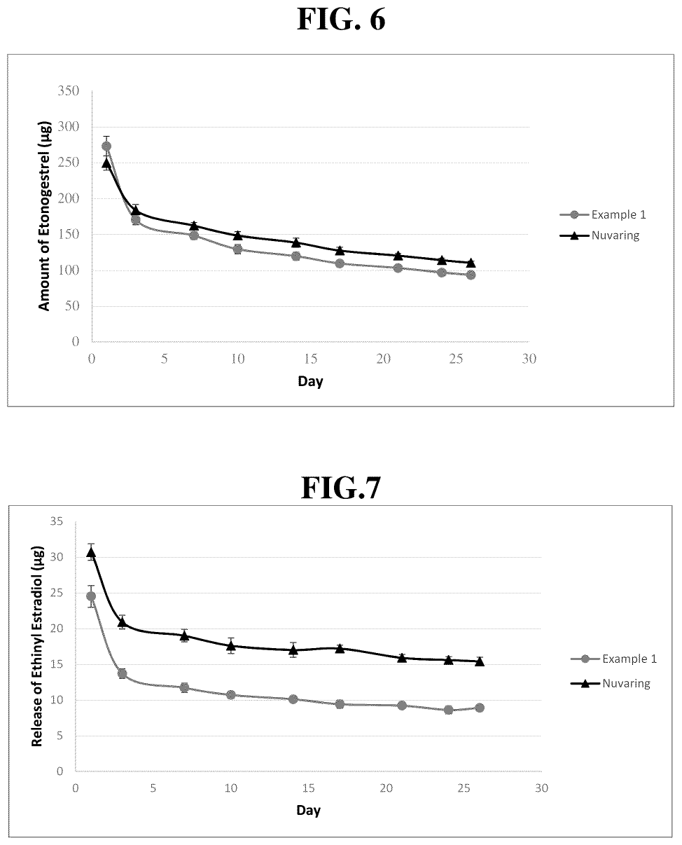

As shown in FIG. 6, the in-vitro release rate of Etonogestrel from vaginal rings produced from Example 1 is compared to the in-vitro release rate of Etonogestrel in the reference product, NuvaRing®. As shown in FIG. 7, the in-vitro release rate of Ethinyl Estradiol from vaginal rings produced from Example 1 is compared to that of reference product, NuvaRing®. The FIG. 6 and FIG. 7 presented the values of the mean of the 12 rings produced from Example 1 and values of the mean of 6 rings of NuvaRing. The vaginal rings produced from Example 1 deliver in-vitro release rates on an average of about 0.12 mg per day of Etonogestrel and 0.010 mg per day of Ethinyl Estradiol for a period of about 21, 24 & 26 days. While the Etonogestrel release rates are about the same for both products, the Ethinyl Estradiol release rate is significantly lower than that of the NuvaRing® reference product. For example, at day 26, the amount of Etonogestrel released by NuvaRing® is on an average of about 109 micrograms, and that of the representative vaginal ring of the invention made by the process of Example 1 is on an average of about 93 micrograms. However, at day 26, the amount of Ethinyl Estradiol released by NuvaRing® is 15.4 micrograms, while that of the system of Example 1 is 8.9 micrograms. The drug delivery system of Example 1 thus releases only 58% of the Ethinyl Estradiol compound compared to the NuvaRing® reference product. The drug delivery systems of this disclosure can release comparable amounts of Etonogestrel to that of reference products while releases a reduced amount of Ethinyl Estradiol. This is significant because the reduced Ethinyl Estradiol amounts will reduce side-effects associated with this hormone, while maintaining a contraceptive prevention effect.

### Example 3. The Pharmacokinetic Profile of Etonogestrel and Ethinyl Estradiol During Use of Representative Vaginal Rings of this Disclosure

The representative vaginal rings of this disclosure exhibit lower Ethinyl Estradiol plasma levels compared to reference products. For the pharmacokinetic profile, Mean Serum Concentration was estimated using an in vitro/in vivo relationship to that of Ethinyl Estradiol and Etonogestrel released from the intravaginal ring composition during one day after administration and the intended use duration. The in vitro-in vivo relationship was established between published serum Etonogestrel and Ethinyl Estradiol levels for NuvaRing and in vitro release testing results obtained for NuvaRing. Then, from in vitro release testing results obtained for the test product using the same analytical method as used for NuvaRing, estimated serum Etonogestrel and Ethinyl Estradiol levels were calculated for the test product. The serum Etonogestrel levels for subjects administered the drug delivery system of Example 1 would be estimated to be comparable based on comparable in vitro release testing results between the drug delivery release system of Example 1 and the NuvaRing as reference. Based on the Ethinyl Estradiol in vitro release testing results for both products, the estimated serum Ethinyl Estradiol level for the test product would be approximately 58% of the value obtained for NuvaRing. The results are summarized in Table 4.

TABLE 4
Estimated Mean Serum Concentrations:

| | Peak serum | |
|---|---|---|
| Drug | concentration (ng/L) | |
| Ethinyl Estradiol | 10 to 15 | ng/L |
| Etonogestrel | 1000 to 1500 | ng/L |

### Example 4: Phase II Clinical Study of Etonogestrel (ENG) and Ethinyl Estradiol (EE) Vaginal Ring in Human Patients

An open-label, randomized, two-period, crossover study will be performed to assess the comparative pharmacokinetics of a representative vaginal ring of the invention compared to the NuvaRing® reference product in healthy adult females.

Objectives: To assess the pharmacokinetics (PK) of etonogestrel (ENG) and ethinyl estradiol (EE) following vaginal ring (28 days) and NuvaRing (28 days) administration, and to assess the safety and tolerability of vaginal ring administered in healthy adult female subjects.

Pharmacokinetic Endpoints: Blood samples are taken at regular time points during each treatment period to determine plasma levels of ENG and EE.

Primary Pharmacokinetic Endpoints: area under the plasma concentration-time curve from time zero to 28 days post dose (AUC0-28d) for ENG; area under the plasma concentration-time curve from time zero to the last measurable concentration (AUC0-last) for ENG; area under the plasma concentration-time curve from time zero to infinity (AUC0-inf) for ENG maximum observed plasma concentration (Cmax) for ENG.

Secondary Pharmacokinetic Endpoints: time to maximum observed plasma concentration (tmax) for ENG; area under the plasma concentration-time curve from time zero to 21 days (AUC0-21d) post dose for ENG; area under the plasma concentration-time curve from time zero to 26 days (AUC0-26d) post dose for ENG; kel, terminal elimination rate constant for ENG; Half-life, T½ for ENG.

Additional Pharmacokinetic Endpoints: area under the plasma concentration-time curve from time zero to 21 days (AUC0-21d) post dose for EE; area under the plasma concentration-time curve from time zero to 26 days (AUC0-26d) post dose for EE; area under the plasma concentration-time curve from time zero to 28 days post dose (AUC0-28d) for EE; area under the plasma concentration-time curve from time zero to infinity (AUC0-inf) for EE; maximum observed plasma concentration (Cmax) for EE; time to maximum observed plasma concentration (tmax) for EE; kel, terminal elimination rate constant for EE; Half-life, T½ for EE.

Safety Endpoints: Safety parameters including adverse events, clinical laboratory parameters, and vital sign measurements.

Overall Study Design: This is an open-label, randomized-sequence, two-period, single-center, crossover, pharmacokinetic study in 40 healthy adult female subjects (18-40 yrs). All subjects receive a vaginal ring (28 days) and NuvaRing (28 days). There is a 28-day washout period after ring removal in Treatment Period 1 and ring insertion in Treatment Period 2. Potential subjects are screened for study eligibility within 28 days before the start of the study on Day 1/Period 1.

Subjects are housed twice during each of the vaginal ring study periods; the first time for ring insertion and the second time for ring removal. Subjects will arrive at the clinical site approximately 12 hours prior to ring insertion. After a supervised overnight fast of at least 10 hours, one vaginal ring is inserted in the morning. Subjects are remained at the research center for collection of blood samples at designated time points and are discharged from the clinical site 24 h after ring insertion. Subjects are returned to the clinical site before each of the subsequent blood samples. On Day 28 the subjects are returned to the clinical site approximately 12 hours prior to ring removal. After a supervised overnight fast of at least 10 hours, a blood sample is collected immediately prior to ring removal (Day 29/672 hour). The ring is removed within 5 min thereafter. Subjects are discharged from the clinic 24 h after ring removal and returned to the clinical site before each of the remaining blood samples. There is a 28-day washout period between treatment periods.

For the calculation of pharmacokinetic parameters, blood samples are collected immediately prior to and after each ring insertion to quantify plasma concentrations of ENG and EE using a validated MS/MS-HPLC bioanalytical method. Safety are assessed by adverse events recording, clinical laboratory and vital signs and tolerability by vaginal examination.

Treatments Administered Test Product: The representative vaginal ring of Example 1 (etonogestrel/ethinyl estradiol vaginal ring) is expected to deliver a mean average of 0.12 mg/0.010 mg per day of ENG/EE respectively.

Reference Products: NuvaRing® (etonogestrel/ethinyl estradiol 11.7/2.7 mg)

Pharmacokinetic Sampling: Blood samples are taken at regular time points during each treatment period as defined as follows: Predose (0), 1, 2, 4, 8, 12 hrs on Day 1, Day 2, Day 3, Day 5, Day 7, Day 9, Day 12, Day 14, Day 18, Day 21, Day 24, Day 26, Day 28, Day 29 (immediately before ring removal), Day 29 (1, 2, 4, 8, 12 hrs post removal), Day 30 (24 hours post-removal), Day 31 (48 hrs post-removal) Day 32 (72 hrs post-removal), Day 33 (96 hours post-removal), Day 34 (120 post-removal hours), Day 35 (144 hours post-removal), Day 36 (168 hours post-removal).

Sample Size Calculations: Sample size calculations are based on Algorta et al. (The European Journal of Contraception & Reproductive Health Care, Volume 22, 2017—Issue 6, Pages 429-438). Using the sample sizes and confidence interval limits for Cmax and AUC0-t for Etonogestrel, the estimated within subject. SD is 0.229 for Cmax and 0.126 for AUC0-t. Assuming a true SD of 0.23 and an assumed true geometric mean ratio in the range from 95% to 105%, a sample size of 33 subjects (rounded to 34) will provide 90% power. A total of 40 subjects are enrolled in this study to account for drop-outs.

Pharmacokinetic Analyses: Pharmacokinetic analysis of EE and ENG are conducted via a noncompartmental analysis using WinNonlin Phoenix (Versions 8.1, Certara, Inc.) as data permit. The following PK parameters are determined: Cmax, Tmax, AUC0-21d, AUC0-26d, AUC0-28d, AUC0-last, AUC0-inf, kel, and T½.

The primary PK endpoints to be derived from the plasma concentration data are Cmax, AUC0-21d, AUC0-28d, and AUC0-inf for ENG. The PK parameters are natural log-transformed prior to the statistical analysis. Comparisons of the PK parameters between treatment groups (A versus B) are carried out using a parametric analysis of variance (ANOVA) model with terms for sequence, period, and treatment as fixed effects, and a random effect of subject within sequence. The treatment difference and the associated two sided 90% confidence interval estimated from the ANOVA model on the log scale will be back-transformed to obtain the estimated ratio of geometric means between treatment groups (A/B) and the two-sided 90% confidence interval for this ratio. NuvaRing (Treatment B) is considered the reference treatment.

Comparative bioavailability of vaginal ring of this disclosure to NuvaRing with respect to ENG is assessed using the two one-sided 90% confidence interval approach with geometric mean ratios for the Cmax, AUC0-28d, and AUC0-inf being contained within (0.80, 1.25).

In addition, PK endpoints for EE will also be determined and include Cmax, AUC0-21d AUC0-28d, and AUC0-inf. To supplement the safety assessments, the systemic exposure of EE following administration of vaginal ring and NuvaRing is statistically evaluated and compared as described above and two-sided 90% confidence intervals are provided. It is expected that the results will show the plasma concentrations of EE and ENG in the subject with the vaginal ring of this disclosure (which comprises a lower amount of EE and ENG) exhibit lower plasma levels of EE compared to the reference product.

### Example 5: Evaluation of Contraceptive Effect of a Representative Vaginal Ring Releasing Ethinyl Estradiol and Etonogestrel on Ovarian Function in Healthy Adult Females

Primary Objective: The primary objective of this study is to evaluate the inhibition of ovulation in Cohort #1 after contraceptive vaginal ring vaginal ring application for 3 treatment cycles.

Secondary Objective: Inhibition of ovulation by measuring (using HPLC-MS) levels of gonadotropins (Follicle Stimulating Hormone (FSH), Luteinizing Hormone (LH), ovarian steroids (Estradiol (E2), Progesterone (P))); Endometrial thickness; Mean maximal follicle size based on measurement of any follicle>1.0 cm in three dimensions; Breakthrough bleeding/spotting assessment; PK of Ethinyl Estradiol (EE), Etonogestrel (ENG) during Cycles 1, 2, and 3; and Pre/post study sex hormone-binding globulin (SHBG) levels.

Primary Endpoints: The primary endpoint is the proportion of subjects in Cohort #1 with complete ovarian inhibition over the entire treatment period (3 treatment cycles). Ovarian inhibition is assessed by rating the suppression of ovaries using the Hoogland score (Hoogland H J, Skouby S O, Contraception, Volume 47, Issue 6, June 1993, Pages 583-590). This score is based on: The follicular size assessed by transvaginal ultrasound (TVUS); and Endogenous hormone levels which include serum E2, and serum P.

Secondary Endpoints: Serum levels of LH, FSH, E2, P; Plasma levels of EE, and ENG.

Blood samples will be taken at regular time points during Cycle 1, 2 and 3 (26-day wear period). Samples for SHBG will be collected pre-insertion on Cycle 1, Day 1 and pre-removal on Cycle 3, Day 27.

Safety parameters including adverse events, clinical laboratory parameters, and vital sign measurements.

Exploratory Endpoints: Subject questionnaire data

Overall Study Design: This is an open-label study to evaluate inhibition of ovulation during treatment with vaginal ring over a period of 3 treatment cycles (approximately 3 months) in healthy female volunteers with a documented ovulatory cycle (pre-treatment cycle). The study will consist of two subject cohorts; Cohort #1 will consist of about 16 healthy women aged 18-35 years with a BMI of ≥18 kg/m² to ≤30 kg/m², Cohort #2 will consist of up to 10 healthy women aged 18-35 years with a BMI of >30 kg/m² to ≤35 kg/m². At screening, all women will go thorough medical and gynecological examination, including a cervical cytology examination (Pap smear*) using the Papanicolaou method and a serum pregnancy test. Eligible subjects will have a pretreatment visit on cycle Day 18 to 21 for confirmation of normal ovulation (P concentration is ≥10 nmol/l). The study is consisted of 3 treatment cycles each consisting of a 26 day ring-insertion period followed by a 2-day ring-free period. *Pap smear test is not done if subject had normal Pap smear test results within 30 days prior to screening visit. Ovarian activity during each treatment cycle is assessed based on follicle size measurements (by TVUS) and serum P and E2 concentrations (6-step grading of ovarian activity according to Hoogland and Skouby (Hoogland H J, Skouby S O, 1993, see above). A Hoogland and Skouby grade 3 and below represents ovulation inhibition. Blood samples are collected for evaluation of gonadotropin levels (FSH, LH) and ovarian steroids (E2, P). In addition, blood samples for analysis of EE and ENG are taken at prespecified sampling times at predose and during each treatment cycle. Samples for SHBG are collected pre-insertion on Cycle 1, Day 1 and pre-removal on Cycle 3, Day 27.

Treatments Administered: Test Product: The representative vaginal ring of Example 1 (etonogestrel/ethinyl estradiol vaginal ring) is expected to deliver a mean average of about 0.12 mg/0.010 mg, respectively, per day.

Pharmacodynamic Sampling Procedures:

Transvaginal Ultrasound (TVUS)

Ovarian activity during each treatment cycle are assessed based on follicle size measurements (by TVUS) and serum P and E2 concentrations. TVUS and serum P and E2 concentrations are performed every 2±1 day starting on Day 4. If a follicle is identified that is of >1.0 cm mean diameter then TVUS and serum P, E2, FSH and LH concentrations are done every day until resolution. TVUS are performed every 2±1 day if there are no follicles>1.0 cm mean diameter.

Any follicle with a diameter>1.0 cm are measured in a frontal view with two dimensions length and height and in a sagittal view with the largest diameter. Mean follicle diameter is the mean of the three measurements.

Endometrial thickness is measured by vaginal ultrasound of the sagittal plane of the uterus and calculated by measuring the sum of both endometrial layers.

Additional Pharmacodynamic Parameters:

Blood samples for determination of FSH, LH, E2 and P serum concentrations are collected at specified time points by the investigator or qualified designee and sent to a laboratory for processing as indicated in the Time and Events Table. The actual date and time of collection for each blood sample are recorded.

Bioanalysis of samples are performed by a qualified clinical laboratory contracted by the sponsor. A currently approved methodology is used to determine serum concentrations of FSH, LH, E2 and P.

Analyses:

The primary endpoint is the proportion of subjects in Cohort #1 with complete ovarian inhibition over the entire treatment period (3 treatment cycles).

Ovarian activity is classified according to Hoogland and Skouby (Hoogland H J, Skouby S O, 1993, see above). Three parameters are combined to a 6-step scoring system: (a) the diameter of the maximum follicle like structure, (b) the E2 serum concentration and (c) the P serum concentration. In each cycle, the highest levels of hormones and the greatest diameter of the largest follicle for each subject will be used to calculate the Hoogland score. The proportion of subjects by Hoogland score are calculated

For each cohort (BMI of ≥18 kg/m² to ≤30 kg/m² and BMI of >30 kg/m² to ≤35 kg/m²) summary statistics (median, minimum, and maximum) for serum hormone levels and vaginal TVUS measurements (follicular diameters and endometrial thickness) are calculated. The median values and ranges are applied to the maximum follicular diameter or the maximum serum hormone level for each subject during each treatment cycle.

Statistical methods for this study consist of descriptive statistics of demographic and baseline data, subject disposition and product exposure along with analyses of PK parameter values, as listed in Table 5.

TABLE 5
Hoogland And Skouby Classification
and Ovarian Activity Grading System

| | | Follicle- | Hormones | |
|---|---|---|---|---|
| Grading of | | like structure | Estradiol | Progesterone |
| ovarian activity | | Size (mm) | (nmol/L) | (nmol/L) |
| 0 | No actiivty | ≤10 | NA | NA |
| 1 | Potential activity | >10 | NA | NA |
| 2 | Non-active FLS | >13 | ≤0.1 | NA |
| 3 | Active FLS | >13 | >0.1 | ≤5 |
| 4 | LUF | >13, persisting | >0.1 | >5 |
| 5 | “Ovulation” | >13, ruptured* | >0.1 | >5 |

FLS—follical-like structure,
LUF—luteinizing unruptured follicle
*Grade 5 will be distinguished from Grade 4 by follicle rupture, defined as the disappearance of the follicle or a decrease in maximum follicle diameter by 50% within 2 to 4 days.

The results are expected to demonstrate the contraceptive effect of the representative vaginal rings of this disclosure.

1. A drug delivery system for use in contraception by administering the system to a subject's vaginal tract, said system comprising:
a) a core comprising ethinyl estradiol and etonogestrel;
b) a membrane covering a portion or all of the core;
wherein, when the system is in the subject's vaginal tract, the system delivers a therapeutically effective dose of the ethinyl estradiol and a therapeutically effective dose of the etonogestrel to the subject for a period of time to produce a contraceptive effect in the subject, and the system delivers less than an average of 0.013 mg per day of the ethinyl estradiol compound to the subject's vaginal tract,
wherein the period of time is about 21 days, about 24 days, about 26 days, about 28 days, or about 3 months after administration to the subject's vaginal tract.

2. The drug delivery system of claim 1, wherein the average is measured after the burst release of the ethinyl estradiol and etonogestrel after administration of the drug delivery system to the subject's vaginal tract.

3. The drug delivery system of claim 1, wherein the amount of etonogestrel in the drug delivery system is from about 1 mg etonogestrel to about 75 mg etonogestrel.

4. The drug delivery system of claim 3, wherein the amount of etonogestrel in the drug delivery system is from about 8 mg to about 9 mg.

5. The drug delivery system of claim 1, wherein the amount of ethinyl estradiol in the drug delivery system is from about 0.1 mg to about 7.5 mg.

6. The drug delivery system of claim 1, wherein the amount of etonogestrel is from about 8 mg to about 9 mg and the amount of ethinyl estradiol is from about 0.1 mg to about 7.5 mg.

7. The drug delivery system of claim 1, wherein the amount of etonogestrel is from 0.4 to 0.7 wt. %.

8. The drug delivery system of claim 1, wherein the amount of ethinyl estradiol in the drug delivery system is from 0.05 to 1.0 wt. %.

9. The drug delivery system according to claim 1, wherein the system releases on an average about 0.1 mg to about 0.2 mg of etonogestrel per day for about 21 days, about 24 days, about 26 days, about 28 days or about 3 months, after the first day of insertion of the drug delivery device to a subject's vaginal tract.

10. The drug delivery system according to claim 1, wherein the system releases on an average from about 0.001 to about 0.013 mg of ethinyl estradiol per day, after the first day of insertion of the drug delivery device to a subject's vaginal tract.

11. The drug delivery system of claim 1, wherein the drug delivery system releases on an average about 0.12 mg of etonogestrel per day, and an average of about 0.010 mg of ethinyl estradiol per day, after the first day of insertion of the drug delivery device to a subject's vaginal tract.

12. The drug delivery system of claim 1, wherein the drug delivery system releases less than 0.027 mg of ethinyl estradiol per day within the burst period after insertion of the drug delivery device to a subject's vaginal tract,
wherein the burst period is the first 24 hr after insertion of the drug delivery device to a subject's vaginal tract.

13. A method of contraception for a total period of time, comprising:
a) retainably positioning the drug-delivery system of claim 1 within a subject's vaginal tract and retaining the system within the subject's vaginal tract for a selected period of time or total period of time; and
b) removing the system from the subject's vaginal tract after the selected period of time or total period of time,
wherein the selected period of time or total period of time is about 21 days, about 24 days, about 26 days, about 28 days, or about 3 months.

14. The method of claim 13, wherein the selected interval of removal is 2 days within a 28-day menstrual cycle.

15. The method of claim 13, wherein the drug delivery system releases on an average about from about 0.001 to about 0.013 mg of ethinyl estradiol per day for about 21 days, about 24 days, about 26 days, about 28 days, or about 3 months after administration to the subject's vaginal tract.

16. The method of claim 13, wherein the drug delivery system releases on an average about 0.1 mg to about 0.2 mg of etonogestrel to the subject for about 21 days, about 24 days, about 26 days, about 28 days, or about 3 months after administration.

17. The method of claim 13, wherein the drug delivery system releases on an average from about 0.001 to about 0.013 mg per day of ethinyl estradiol and on an average from about 0.10 to about 0.12 mg of etonogestrel per day for about 21 days, about 24 days, about 26 days, about 28 days, or about 3 months after administration to the subject's vaginal tract.

18. The method of claim 13, wherein the drug delivery system releases less than 0.027 mg of ethinyl estradiol per day within the burst period after insertion of the drug delivery device to a subject's vaginal tract.

19. The method of claim 18, wherein the burst period is the first 24 hr after insertion of the drug delivery device to a subject's vaginal tract.

20. The method of claim 13, wherein after retainably positioning the drug-delivery system of claim 1 within a subject's vaginal tract, the subject's serum concentration of ethinyl estradiol is in the range of 10-15 ng/L.

21. The drug delivery system of claim 1, wherein the core and the membrane are each independently made of a thermoplastic polymer or an elastomer,
wherein the core is made of an ethylene-vinyl acetate copolymer having a vinyl acetate relative molar content from 25 to 35%, and
wherein the membrane is made of an ethylene-vinyl acetate copolymer having a relative molar content from 8 to 10%.

22. The drug delivery system of claim 21, wherein the core comprises ethylene-vinyl acetate copolymer from about 81 wt. % to about 92 wt. % relative to the total weight of the drug delivery system.

23. The drug delivery system of claim 21, wherein the membrane comprises ethylene-vinyl acetate copolymer from about 11.7 wt. % to about 14.3 wt. % relative to the total weight of the drug delivery system.
